(19) United States
(12) Patent Application Publication
(10) Pub. No.: US 2010/0130549 A1
(43) Pub. Date: May 27, 2010

(54) PSYCHOTROPIC COMPOUNDS, COMPOSITIONS AND METHODS OF USE

(75) Inventors: Irit Gil-Ad, Herzliya (IL); Moshe Portnoy, Ramat-Hasharon (IL); Avraham Weizman, Tel-Aviv (IL); Liat Lomnitski, Givat Shmuel (IL)

Correspondence Address: LERNER, DAVID, LITTENBERG,;KRUMHOLZ & MENTLIK, 600 SOUTH AVENUE WEST, WESTFIELD, NJ, 07090, US

(73) Assignee: RAMOT AT TEL-AVIV UNIVERSITY LTD., Tel Aviv (IL)

(21) Appl. No.: 12/522,637

(22) PCT Filed: Jan. 10, 2008
(86) PCT No.: PCT/IL2008/000049

§ 371 Date: Jan. 8, 2010

Related U.S. Application Data
(60) Provisional application No. 60/884,303, filed on Jan. 10, 2007.

(51) Int. Cl. A61K 31/4525; A61K 31/135; A61P 25/00; A61P 35/00

(52) U.S. Cl. 514/321; 514/657

(57) ABSTRACT

The present invention provides a method for treating or preventing a disease or disorder treatable by the inhibition of serotonin reuptake in a patient, and/or norepinephrine reuptake and/or dopamine reuptake in a patient, the method comprising administering to the patient a neurotransmitter reuptake inhibiting-effective amount of at least one compound of the formula A-L-B (I) or a pharmaceutically acceptable salt, ester or prodrug thereof, wherein A is a psychotropic derivative; L is a linking group comprising two carbon atoms; and B is an alkyl, alkenyl, alkynyl or aralkyl comprising at least one substituent of the formula Q, wherein: the alkyl, alkenyl, alkynyl or aralkyl is optionally substituted with one or more halogean halogens, hydroxyl, cyano, nitro, amino or thiol; and Q is OR⁶, OC(O)R⁶, C(O)R⁶, C(S)R⁶, CO2R⁶, C(O)SR⁶, C(O)NR⁶R₇, C(S)NR⁶R⁷, NR⁶R₇, NR₆C(O)R₇, NR⁶C(S)R₇, NR⁶C(O)NR⁷R⁸, NR6C(S)NR⁷R⁸, NR⁶SO2R⁷, NR⁶SO2NR⁷R⁸, SR⁶, SC(O)R⁶, SC(O)NR⁶R⁷, S(O)R⁶, SO2R⁶, SO2NR⁶R⁷, or NR⁶SO2NR⁷R⁸.

### BACKGROUND OF THE INVENTION

Psychotropics include drugs or agents that are typically employed for the therapy of psychiatric disorders such as schizophrenia and mood disorders. In general, psychotropic drugs interact with central and peripheral neurotransmitters and their receptors, such as serotoninergic, dopaminergic, α-adrenergic, cholinergic etc. Selective serotonin reuptake inhibitors (SSRIs), such as paroxetine, sertraline, and fluoxetine, are among the most commonly prescribed antidepressants and are considered highly effective and relatively safe. Other antidepressant agents are the tricyclic group of agents (TCA) (e.g imipramine, amitryptiline, clomipramine, nortryptiline), the serotonin noradrenaline reuptake inhibitors (NARI) (e.g venlafaxine and duloxetine), the noradrenaline reuptake inhibitor (reboxetine) and the atypical agents (e.g mianserin, mirtazapine, nefazodone, and trazodone). SSRI antidepressants are used to treat a variety of diseases and disorders. For example, SSRIs are used for the treatment or prophylaxis of disease or disorders such as posttraumatic stress disorders (PTSD), obsessive compulsive disorders, anxiety, panic attacks, pain, neuralgic pain, postherpetic neuralgia, phobias of various types, and eating disorders, to name a few. (Shiloh, Nutt, Weizman Atlas Psychiatric Pharmacotherapy, antidepressants 26-36, editor Martin Dunitz LTD, London UK 1999, Psychotropics, Lundbeck Institute 2002-3 edition, antidepressants 239-324 Denmark).

An important aspect of psychotropic agents is the ability of these agents to interact with CNS neurotransmitters receptors. The most important receptors are the monoamine receptors such as the dopaminergic (DA), noradrenergic(NE) and the serotoninergic (5HT) receptors. Each of these neurotransmitter's systems is divided into subtype receptors which are located at different areas of the brain and each possesses a specific function. In addition to the three main biogenic amine receptors, there are clusters of other important receptors in the brain such as: adrenergic, muscarinic, GABAergic, glutamatergic receptors. Depression for example, is associated with decreased serotonin and noradrenalin at post synaptic receptors. Presynaptic plasma membrane transporters for 5-HT (SERT), NE (NET), and DA (DAT), control synaptic actions of these monoamines by rapidly clearing the released amine. Monoamine transporters are the sites of action for widely used antidepressants and present high affinity molecular targets for drugs development. (Jayanthi et al., Regulation of Monoamine Transporters: Influence of Psychostimulants and Therapeutic Antidepressants, AAPS J. 27, 7(3):E728-38 (2005)). Inhibition of SERT and/or NET is generally associated with antidepressant activity. (White K J et al. Serotonin transporters: implications for antidepressant drug development, AAPS J. 7(2):E421-33 (2005)). Recently DA was suggested to possess a role in depression. (Jiao et al., Antidepressant drug induced alterations in binding to central dopamine transporter sites in the Wistar Kyoto rat strain. Progress in Neuro-Psychopharmacology and Biological Psychiatry, 30, 30-41 (2006)). In addition to depression, the monoamine transporters are involved in many psychiatric and other disorders such as suicide, impulsive violence, alcoholism, anxiety, PTSD, OCD, pain sensation, affective states, learning and memory, endocrine, and autonomic functions, movement disorders, emotion, and cognition.

Potential CNS agents are often first assessed using a series of animal models for certain psychiatric states such as depression and anxiety. With regard to anxiety, chronically administered antidepressant drugs, particularly selective serotonin (5-HT) reuptake inhibitors (SSRIs), are clinically effective in the treatment of all anxiety disorders such as major depressive disorder (MDD), generalized anxiety disorder (GAD), panic disorder (PD), social anxiety disorder (SAD), post-traumatic stress disorder (PTSD), and obsessive-compulsive disorder (OCD). The clinical effectiveness of traditional anxiolytics, such as benzodiazepines (BDZs), is limited to generalized anxiety disorder or acute panic attacks. Although animal models of anxiety are sensitive to BDZs, few respond to antidepressants. (Borsinil et al., Do animal models of anxiety predict anxiolytic-like effects of antidepressants? Psychopharmacology, 1155 (2002)).

A need exists for new CNS active agents and methods of using such compounds for treating psychiatric diseases or disorders. The present invention provides such compounds and methods.

### BRIEF SUMMARY OF THE INVENTION

In one embodiment, the present invention provides a method for treating or preventing a disease or disorder treatable by the inhibition of serotonin reuptake, and/or norepinephrine reuptake and/or dopamine reuptake or an agent that interferes in. another way with brain neurotransmitters activity in a patient, which method includes administering to the patient a neurotransmitter reuptake inhibiting-effective amount or other neurotransmitter modulating activity effective amount of at least one compound of the formula A-L-B (I), wherein A is represented by the formulae (A1), (A2), or (A3):

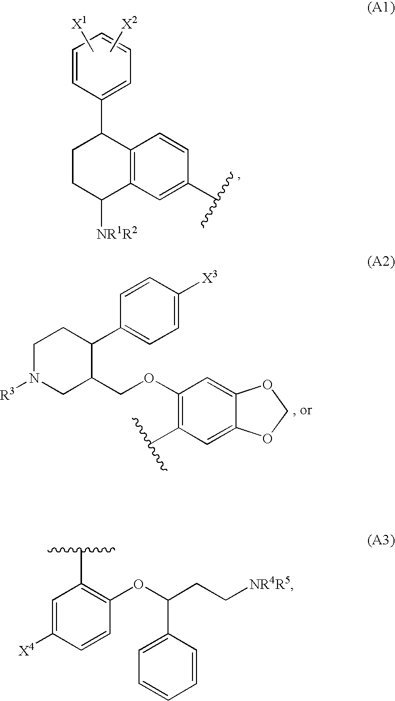

wherein: R¹, R², R³, R⁴ and R⁵ are the same or different and each is independently a hydrogen or a C₁₋₆ alkyl; X¹ and X² are the same or different and each is independently a hydrogen, a halogen, a C₁₋₆ haloalkyl, a C₁₋₆ alkoxy, or a cyano; X³ is a hydrogen, a C₁₋₆ alkyl, a C₁₋₆ alkoxy, a C₁₋₆ haloalkyl, a hydroxyl, a halogen, a C₁₋₆ alkylthio, or an aryl(C₁₋₆)alkoxy; X⁴ is a halogen, a C₁₋₆ haloalkyl, a C₁₋₆ alkyl, a C₁₋₆ alkoxy, or a C₂₋₆ alkenyl; L is a linking group comprising two carbon atoms; and B is an alkyl, alkenyl, alkynyl or aralkyl comprising at least one substituent of the formula Q, wherein the alkyl, alkenyl, alkynyl or aralkyl is optionally substituted with one or more halogens, hydroxyl, cyano, nitro, amino, or thiol, and Q is OR⁶, OC(O)R⁶, C(O)R⁶, C(S)R⁶, CO₂R⁶, C(O)SR⁶, C(O)NR⁶R⁷, C(S)NR⁶R⁷, NR⁶R⁷, NR⁶C(O)R⁷, NR⁶C(S)R⁷, NR⁶C(O)NR⁷R⁸, NR⁶C(S)NR⁷R⁸, NR⁶SO₂R⁷, NR⁶SO₂NR⁷R⁸, SR⁶, SC(O)R⁶, SC(O)R⁶, SC(O)NR⁶R⁷, S(O)R⁶, SO₂R⁶, SO₂NR⁶R⁷, or NR⁶SO₂NR⁷R⁸, wherein R⁶, R⁷, and R⁸ are the same or different and each is independently a hydrogen, a C₁₋₆ alkyl, an aryl, an aralkyl, or a pharmaceutically acceptable solubility modifying group; or a salt, ester, or prodrug of the compound, which may include, e.g. a pharmaceutically acceptable salt, ester, or the like. It will be appreciated that the compound of the formula A-L-B (I) includes geometric and optical isomers thereof, e.g., diasteomers and mixtures thereof, enantiomers (e.g., a substantially pure enantiomer or an enantiomeric mixture), and molecules of the same general formula having any other suitable combination of chiral centers. The compound of the formula A-L-B (I) also includes, e.g., solvates, hydrates and polymorphs thereof.

In addition A can be represented by monocyclic antidepressants (e.g. fluoxetine, reboxetine, tomoxetine, duloxetine), other bicyclic (e.g. sertraline, paroxetine, idazoxan, dapoxetine, tricyclic (e.g. desipramine, clominpramine, amitriptyline, doxepine) and tetracyclic antidepessants (e.g. mianserin) which can undergo derivatization via similar synthetic schemes.

In accordance with the method of the present invention, the compounds can be administered as a pharmaceutical composition comprising a pharmaceutically acceptable carrier and a therapeutically effective amount of at least one compound of the formula A-L-B (I), as described herein.

In a preferred embodiment, the disease or disorder is a psychiatric disorder, e.g., a psychiatric disorder associated with abnormal serotonin reuptake or a neurological disorder such as a neurodegenerative disease and dementia.

Exemplary psychiatric disorders can include, e.g., psychiatric disorders associated with cancer.

### BRIEF DESCRIPTION OF THE DRAWINGS

FIG. 1A illustrates the effect of an exemplary compound and sertraline given ip on distance moved in BALB/c mice in the open field test.

FIG. 1B illustrates the effect of an exemplary compound and sertraline given ip on velocity in BALB/c mice in the open field test.

FIG. 1C illustrates the effect of an exemplary compound and sertraline given ip on duration in the center for BALB/c mice in the open field test.

FIG. 1D illustrates the effect of an exemplary compound and a comparative compound, sertraline given ip, on the ratio of total duration between zone 2+3 to zone 1 (wherein zone 1 is the periphery zone 2 the medium and zone 3 the center of the open field) for BALB/c mice.

FIG. 2A illustrates the effect of an exemplary compound at 3 different doses (2.5, 7.5 and 22.5 mg/kg given orally on distance moved in BALB/c mice in the open field test.

FIG. 2B illustrates the effect of an exemplary compound at 3 different doses (2.5, 7.5 and 22.5 mg/kg given orally on velocity in BALB/c mice in the open field test.

FIG. 2C illustrates the effect of an exemplary compound at 3 different doses (2.5, 7.5 and 22.5 mg/kg given orally on total duration in center (zone 2 and zone 3) for BALB/c mice in the open field test.

FIG. 3A illustrates the effect of an exemplary compound at 3 different doses (2.5, 7.5 and 22.5 mg/kg given orally on distance moved in BALB/c mice in the open field test.

FIG. 3B illustrates the effect of an exemplary compound at 3 different doses (2.5, 7.5 and 22.5 mg/kg given orally on velocity in BALB/c mice in the open field test.

FIG. 3C illustrates the effect of an exemplary compound at 3 different doses (2.5, 7.5 and 22.5 mg/kg given orally on frequency to the center (zone3) in the open field.

FIG. 4A illustrates the effect of an exemplary compound and a comparative compound, sertraline, given ip on frequency to arms in the elevated plus maze in BALB/c mice.

FIG. 4B illustrates the effect of an exemplary compound and a comparative compound, sertraline, given ip on the duration (min) in the different arms in the elevated plus maze with BALB/c mice (total duration).

FIG. 5A illustrates the effect of an exemplary compound at 3 different doses (2.5, 7.5 and 22.5 mg/kg given orally on the elevated plus maze with BALB/c mice (frequency to zone).

FIG. 5B illustrates the effect of an exemplary compound at 3 different doses (2.5, 7.5 and 22.5 mg/kg given orally on total duration in the different arms in the elevated plus maze in BALB/c mice.

FIG. 6A illustrates the effect of an exemplary compound at 3 different doses (2.5, 7.5 and 22.5 mg/kg given ip in the forced swim test on distance moved in BALB/c mice.

FIG. 6B illustrates the effect of an exemplary compound at 3 different doses (2.5, 7.5 and 22.5 mg/kg given ip in the forced swim test on velocity in BALB/c mice.

FIG. 6C illustrates the effect of an exemplary compound at 3 different doses (2.5, 7.5 and 22.5 mg/kg given ip in the forced swim test on strong mobility on BALB/c mice.

FIG. 6D illustrates the effect of an exemplary compound at 3 different doses (2.5, 7.5 and 22.5 mg/kg given ip in the forced swim test on immobility in BALB/c mice.

FIG. 7A illustrates the effect of sertraline given ip in the forced swim test on strong mobility on BALB/c mice.

FIG. 7B illustrates the effect of sertraline given ip in the forced swim test on immobility on BALB/c mice.

FIG. 8A illustrates the effect of an exemplary compound at 3 different doses (2.5, 7.5 and 22.5 mg/kg given ip on distance moved in BALB/c mice in the forced swim test.

FIG. 8B illustrates the effect of an exemplary compound at 3 different doses (2.5, 7.5 and 22.5 mg/kg given ip on distance moved in BALB/c mice in the forced swim test.

FIG. 8C illustrates the effect of an exemplary compound at 3 different doses (2.5, 7.5 and 22.5 mg/kg given i.p on immobility in BALB/c mice in the forced swim test.

FIG. 9 illustrates the effect of an exemplary compound and a comparative compound, sertraline, on motor activity using the rotor rod test in BALB/c mice.

FIG. 10 illustrates the acute effect of an exemplary compound 10-30 mg/kg given ip and a comparative compound, sertraline, on body weight during 12 days in ICR male mice.

FIG. 11A illustrates the effect that acute oral administration of an exemplary compound (25-100 mg/kg) has on the weight of ICR male mice.

FIG. 11B illustrates the effect of oral administering of an exemplary compound on stimulation (number of rearings 24-168 hr post treatment) in ICR male mice.

FIG. 12 illustrates the effect of subchronic administration (3×1/week for 2 weeks) of an exemplary compound or equimolar dose of sertraline on body weight of ICR male mice. Histopathology evaluation of heart, spleen, liver and kidney of the subchronic treated animals showed that at the high dose of 30 mg/kg ip, both sertraline and the compound of formula (Ia) induced a minimal to mild irritation at the capsule of the abdominal organs. Except for this local irritation no other toxicity was noted.

FIG. 13 illustrates the effect of an exemplary compound (oral 10 mg/kg) and a comparative compound, sertraline, on latency to responding to heating in the hot plate test.

### DETAILED DESCRIPTION OF THE INVENTION

The present invention provides a method for treating or preventing a disease or disorder treatable by the inhibition of serotonin reuptake and possibly NE norepinephrine reuptake and/or dopamine reuptake and/or other neurotransmitter Modulators in a patient, the method comprising administering to the patient a serotonin reuptake inhibiting-effective amount of at least one compound of the formula A-L-B (I), wherein A is represented by the formulae (A1), (A2), or (A3):

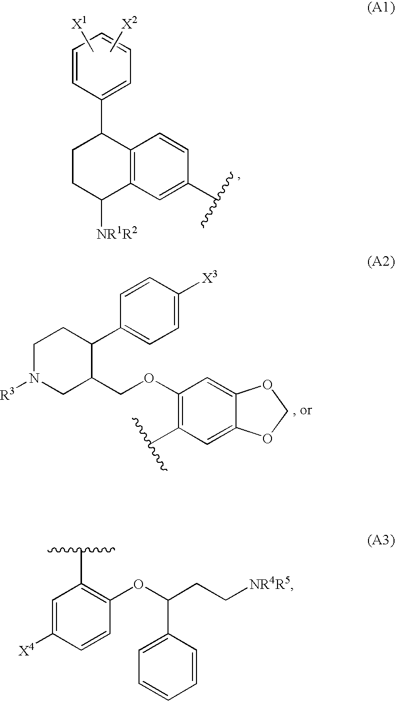

wherein:
R¹, R², R³, R⁴ and R⁵ are the same or different and each is independently a hydrogen or a C₁₋₆ alkyl,
X¹ and X² are the same or different and each is independently a hydrogen, a halogen, a C₁₋₆ haloalkyl, a C₁₋₆ alkoxy, or a cyano,
X³ is a hydrogen, a C₁₋₆ alkyl, a C₁₋₆ alkoxy, a C₁₋₆ haloalkyl, a hydroxyl, a halogen, a C₁₋₆alkylthio, or an aryl(C₁₋₆)alkoxy, and
   X⁴ is a halogen, a C₁₋₆ haloalkyl, a C₁₋₆ alkyl, a C₁₋₆ alkoxy, or a C₂₋₆ alkenyl; L is a linking group comprising two carbon atoms; and B is an alkyl, alkenyl, alkynyl or aralkyl comprising at least one substituent of the formula Q, wherein the alkyl, alkenyl, alkynyl or aralkyl is optionally substituted with one or more halogens, hydroxyl, cyano, nitro, amino, or thiol; Q is OR⁶, OC(O)R⁶, C(O)R⁶, C(S)R⁶, CO₂R⁶, C(O)SR⁶, C(O)NR⁶R⁷, C(S)NR⁶R⁷, NR⁶R⁷, NR⁶C(O)R⁷, NR⁶C(S)⁷, NR⁶C(O)NR⁷R⁸, NR⁶C(S)NR⁷R⁸, NR⁶SO₂NR⁷R⁸, SR⁶, SR(O)R⁶, SC(O)NR⁶R⁷, S(O)R⁶, SO₂R⁶, SO₂NR⁶R⁷, or NR⁶SO₂NR⁷R⁸, wherein R⁶, R⁷, and R⁸ are the same or different and each is independently a hydrogen, a C₁₋₆ alkyl, an aryl, an aralkyl, or a pharmaceutically acceptable solubility modifying group; or a salt, ester, or prodrug thereof, which may include, e.g., a pharmaceutically acceptable salt, ester, etc.

In one embodiment, B is —(CH₂)ₙ(CHR⁹)ₘQ, —Ar(CH₂)ₙ(CHR⁹)ₘQ, —(CH₂)ₙAr(CHR⁹)ₘQ or —(CH₂)ₙ(CHR⁹)ₘArQ, wherein m and n are the same or different and each is independently an integer of from 0 to about 6, provided that m and n are not both zero when B is —(CH₂)ₙ(CHR⁹)ₘQ; Ar is a bivalent aryl (which is covalently bonded to the L and the —(CH₂)ₙ, e.g., in —Ar(CH₂)ₙ(CHR⁹)ₘQ, covalently bonded to the —(CH₂)ₙ and the (CHR⁹)ₘ in —(CH₂)ₙAr(CHR₉)ₘQ, covalently bonded to the (CHR⁹)ₘ and the Q in —(CH₂)ₙ(CHR⁹)ₘArQ, etc.); R⁹ is a hydrogen, a C₁₋₆ alkyl, or an aryl; and the Ar, (CH₂)ₙ and (CHR⁹)ₘ are optionally substituted with one or more halogens, hydroxyl, cyano, nitro, amino, or thiol. For example, B can be —(CH₂)ₙQ, —(CH₂)ₙArQ or —Ar(CH₂)ₙQ, wherein n is from 0 to about 6 provided that n is not zero when B is —(CH₂)ₙQ. In a preferred embodiment, B is —(CH₂)ₙQ, n is about 3, and/or Q is OR⁶, OC(O)R⁶, NR⁶R⁷, SR⁶ or SC(O)R⁶.

In another embodiment, B is —(CH₂)ₙ(CHR⁹)ₘQ, —Ar(CH₂)ₙ(CHR⁹)ₘQ, —(CH₂)ₙAr(CHR⁹)ₘQ or —(CH₂)ₙ(CHR⁹)ₘArQ, wherein m and n are the same or different and each is independently from 0 to about 6 provided that m and n are not both zero when B is —(CH₂)ₙ(CHR⁹)ₘQ; Ar is a bivalent aryl; R⁹ is a hydrogen, a C₁₋₆ alkyl, or an aryl; and the Ar, (CH₂)ₙ and (CHR⁹)ₘ are optionally substituted with one or more halogens, hydroxyl, cyano, nitro, amino, or thiol. In a preferred embodiment, B is —(CH₂)ₙQ. In a more preferred embodiment, B is —(CH₂)ₙQ, wherein n is about 3.

Exemplary compounds administered in accordance with the present invention include compounds of the formula A-L-B (I) as described herein, wherein Q is OR⁶, OC(O)R⁶, NR⁶R⁷, SR⁶ or SC(O)R⁶. Exemplary compounds administered in accordance with the present invention also include compounds of the formula A-L-B (I) as described herein, wherein R⁶, R⁷ and R⁸ are hydrogen and/or wherein n (in the linker B as defined herein) is from 2 to 4.

L can include any suitable linking group comprising two carbon atoms. For example, L can include a linker (e.g., a two-carbon linker) comprising carbon-carbon single bond, a linker (e.g., a two-carbon linker) comprising a carbon-carbon double bond, or a linker (e.g., a two-carbon linker) comprising a carbon-carbon triple bond. In one embodiment, L is a carbon-carbon triple bond. In some embodiments, L is a carbon-carbon triple bond, and R⁶, R⁷, and R⁸ are hydrogen. In other embodiments, L is a carbon-carbon triple bond, and n is from 2 to 4.

As utilized herein, the term “alkyl” generally includes straight-chain and branched-chain alkyl radicals, preferably containing from 1 to about 6 carbon atoms. Examples of alkyl substituents include methyl, ethyl, propyl, isopropyl, n-butyl, sec-butyl, isobutyl, tert-butyl, pentyl, isoamyl, hexyl, and the like.

The term “alkenyl” generally includes straight-chain and branched-chain alkenyl radicals having one or more carbon-carbon double bonds and preferably containing from about 2 to about 6 carbon atoms. Examples of alkenyl substituents include vinyl, allyl, 1,4-butadienyl, isopropenyl, and the like.

The term “alkynyl” generally includes straight-chain and branched-chain alkynyl radicals having one or more carbon-carbon triple bonds and preferably containing from about 2 to about 6 carbon atoms. Examples of alkynyl substituents include ethynyl, propynyl (propargyl), butynyl, and the like.

The term “alkoxy” generally includes alkyl ether radicals, wherein the term “alkyl” is as defined herein. Examples of alkoxy radicals include C₁₋₆ alkoxy, such as methoxy, ethoxy, n-propoxy, isopropoxy, n-butoxy, isobutoxy, sec-butoxy, tert-butoxy, hexanoxy, and the like.

The term “alkylthio” generally includes alkyl thioether radicals, wherein the term “alkyl” is as defined herein. Examples of alkylthio radicals include C₁₋₆ alkthio, such as methylthio (SCH₃), ethylthio (SCH₂CH₃), n-propylthio, isopropylthio, n-butylthio, isobutylthio, sec-butylthio, tert-butylthio, n-hexylthio, and the like.

The term “aryl” refers to an aromatic carbocyclic radical, as commonly understood in the art, and includes monocyclic and polycyclic aromatics such as, for example, phenyl and naphthyl radicals.

The term “aralkyl” refers to an alkyl, as defined herein, substituted with one or more aryl moieties as defined herein. Preferably, the alkyl portion of the aralkyl is a C₁₋₆ alkyl, wherein at least one hydrogen atom of the C₁₋₆ alkyl moiety is replaced by at least one aryl substituent. Examples thereof include benzyl, 1-phenethyl, 2-phenethyl, 3-phenylpropyl, 2-phenyl-l-propyl, and the like.

The term “aryl(C₁₋₆)alkoxy” generally includes C₁₋₆ alkoxy substituents, as defined herein, substituted with one or more aryls as defined herein. Examples of aralkoxy substituents include phenylmethoxy, 2-phenylethoxy, 2-phenyl-1-propoxy, and the like.

The term “haloalkyl” generally includes alkyl substituents, as defined herein, substituted with one or more halogen atoms. Examples of haloalkyl radicals include: C₁₋₆ fluorinated alkyl, such as trifluoromethyl, pentafluoroethyl, 2-fluoroethyl, 2-fluoro-1-propyl, and the like; C₁₋₆ chlorinated alkyl, such as chloromethyl, 2-chloroethyl, 2-chloro-1-propyl, and the like; C₁₋₆ brominated alkyl, such as bromomethyl, 2-bromoethyl, 2-bromo-1-propyl, and the like; C₁₋₆ iodinated alkyl, such as iodomethyl, 2-iodoethyl, 2-iodo-1-propyl, and the like.

The term “solubility modifying group” generally includes substituents that are useful in the art for modifying the solubility of a compound. It will be appreciated that the “solubility modifying group” can alter the molecular weight and lipophilicity of a particular compound, which, in turn, can impact the bioavailability of a particular compound, modulate or control tissue distribution, modify the ability of a particular compound to penetrate the blood-brain barrier, facilitate penetration into the skin for topical applications and/or facilitate systemic administration by a transdermal administration, and the like. The “solubility modifying group” can be charged or neutral and can be lipophilic or hydrophilic Exemplary “solubility modifying groups” include, polyalcohols (e.g., polyethylene glycol having from about 2 to 25 units), polyol ethers, copolymers of ethylene and propylene glycol, esters of polyethylene glycols (e.g., laurate esters of polyethylene glycols), triphenylmethyl, naphthylphenylmethyl, palmitate, distearylglyceride, didodecylphosphatidyl, cholesteryl, arachidonyl, octadecanyloxy, tetradecylthio, alkyl groups, aryl groups, heteroaryl groups, hydroxyacids (e.g., lactic acid), amino acids, and the like. It will be appreciated that the solubility modifying group (and other substituents on the molecule) can be employed to inhibit (or even prevent) transport of the molecule across the blood-brain barrier, e.g., to minimize any psychotropic side effects that may be associated with the parent molecule.

Prodrugs of the compound administered in accordance with the method of the present invention can include derivatives or analogs of the type that are used as, or understood in the art to be useful as, prodrugs of biologically active compounds. The prodrugs may themselves be active or inactive and, by virtue of chemical or enzymatic attack, can be converted to the parent drug in vivo before or after reaching a particular site of action. Prodrugs can include derivatives such as, e.g., esters and the like, which can be prepared, e.g., by reacting the active compound with a suitable acylating agent if the active compound includes a suitably reactive alcohol functional group. Prodrugs also can include carrier-linked prodrugs, bioprecursors, and the like. A carrier-linked prodrug, for example, can result from a temporary linkage of the active molecule with a transport moiety. Such prodrugs typically are less active or inactive relative to the parent active drug. The transport moiety can be chosen for its non-toxicity and its ability to ensure the efficient release of the active principle. A bioprecursor can result from a molecular modification of the active drug itself, e.g., by generation of a new molecule that is capable of acting as a substrate for one or more metabolizing enzymes whereby the action of a metabolizing enzyme produces the active drug in vivo. See also WO 2006/0046967.

It will be appreciated that prodrugs can be employed to alter a variety of properties, including drug pharmacokinetics, stability, solubility, toxicity, specificity, duration of the pharmacological effect of the drug, and the like. By altering pharmacokinetics, the drug bioavailability can be increased, e.g., by increasing absorption, modulating distribution (e.g., systemically or in one or more particular tissues), controlling biotransformation, controlling the rate excretion of the drug, reducing acute toxicity, and the like. It is well within the skill of an ordinarily skilled artisan to design an develop a suitable prodrug of a particular biologically active molecule. In designing such prodrugs, factors taken into consideration can include, for example, the type of linkage that exists between the carrier and the drug (typically a covalent bond), the biological activity or toxicity of the prodrug relative to the active principle, the cost of preparing the prodrug, ease of synthesis, the reversibility of conversion to the active principle, and the like. Prodrugs may be prepared, e.g., by forming an ester, hemiester, carbonate ester, nitrate ester, amide, hydroxamic acid, carbamate, imine, mannich base, enamine, and the like. Prodrugs also may be prepared by functionalizing an active agent with an azo, a glycoside, a peptide, an ether, and the like, or by forming a salt, a complex, a phosphoramide, an acetal, a hemiacetal, a ketal, and the like.

In one embodiment, the compound associated with the method of the present invention is of the formula A-L-B (I), wherein A is represented by formula (Al), and X¹ and X² are the same or different and each is independently a halogen. Alternatively or additionally, when A is of the formula (A1), R¹ and R² can be the same or different wherein each is independently a hydrogen or a methyl, e.g., wherein one of R¹ and R² is a methyl and the other is a hydrogen, or wherein both R¹ and R² are hydrogen or methyl. An exemplary substituent of the formula (A1) is represented by formula:

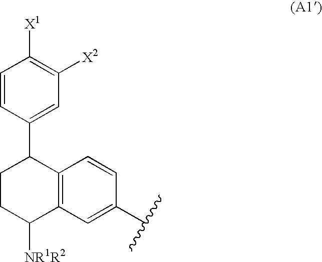

wherein X¹ and X² are the same or different and each is halogen, and R¹ and R² are the same or different and each is independently a hydrogen or a methyl. When A is of the formula (A1′), X¹ and X² preferably are chlorine, and one of R¹ and R² is a hydrogen and the other is a methyl.

In another embodiment, the compound administered in accordance with the method of the present invention is of the formula A-L-B (I), wherein A is represented by formula (A2). When A is of the formula (A2), R³ preferably is hydrogen. Alternatively or additionally, when A is of the formula (A2), X³ preferably is a hydrogen or a halogen, and is more preferably a halogen (e.g., fluorine). In one series, when A is represented by formula (A2), R³ is a hydrogen and X³ is a halogen (which is preferably fluorine).

In yet another embodiment, the compound administered in accordance with the method of the present invention is of the formula A-L-B (I), wherein A is represented by formula (A3). When A is of the formula (A3), R⁴ and R⁵ are the same or different and each can be, e.g., independently a methyl or a hydrogen. For instance, when A is of the formula (A3), one of R⁴ and R⁵ can be a methyl and the other can be a hydrogen. Alternatively or additionally, when A is of the formula (A3), X⁴ can be a C₁₋₆ haloalkyl, such as for example, C₁₋₆ fluorinated alkyl (e.g., trifluoromethyl). In one series, when A is of the formula (A3), one of R⁴ and R⁵ is methyl and the other is hydrogen, and X⁴ is trifluoromethyl.

It will be appreciated that the compound of the formula A-L-B (I) includes geometrical and optical isomers, e.g., diasteomers and diastereomeric mixtures, enantiomers (e.g., a substantially pure enantiomer or an enantiomeric mixture), and molecules of the same general formula having any other suitable combination of chiral centers. For instance, A can include substituents of the formulae:

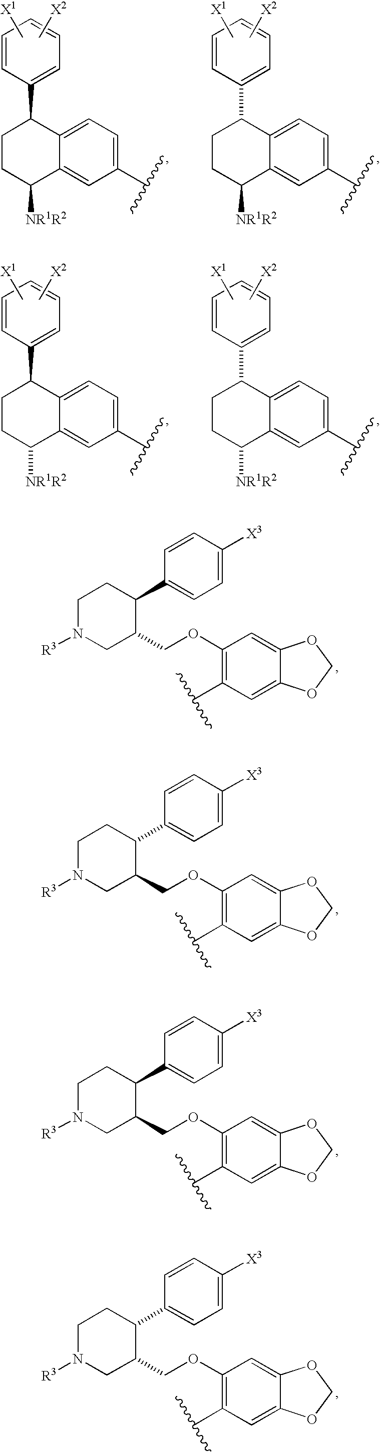

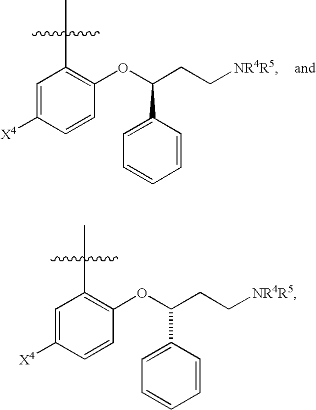

and combinations thereof, wherein X¹-X³ and R¹-R⁵ are as define herein.

The compound of the formula A-L-B (I) also includes, e.g., solvates, hydrates and polymorphs.

Exemplary compounds that can be administered in accordance with the method of the present invention include the following:

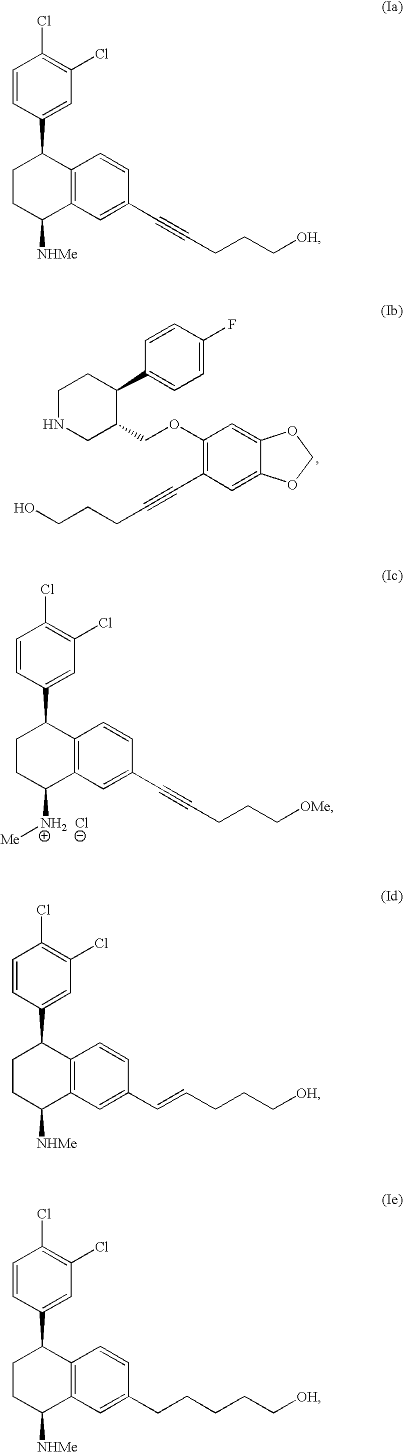

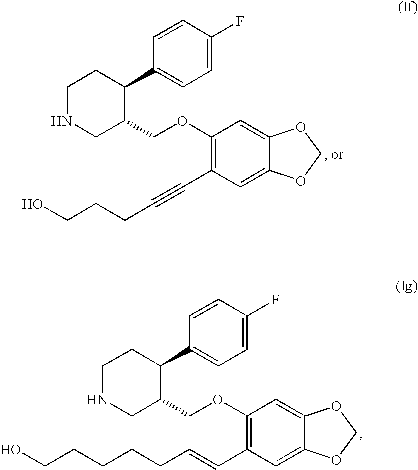

and pharmaceutically acceptable salts, esters, and prodrugs thereof. Particularly preferred compounds include the following:

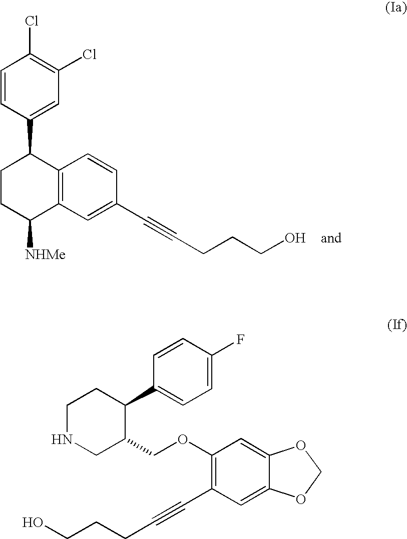

and pharmaceutically acceptable salts, esters, and prodrugs thereof.

In accordance with the method of the present invention, compounds of formula (I) can be administered as a pharmaceutical composition, which includes a pharmaceutically acceptable carrier and a therapeutically effective amount of the compound. The therapeutically effective amount preferably is a serotonin reuptake-inhibiting effective amount, which can include, for example, an amount of one or more compounds of formula (I) sufficient to therapeutically inhibit abnormal or undesirable serotonin reuptake in a particular patient, e.g., an anti-psychotic effective amount. The therapeutically effective amount preferably includes the dose necessary to achieve an “effective level” of one or more of the active compounds in an individual patient. The effective level can be defined, for example, as the amount required in an individual patient to achieve a serotonin reuptake-inhibiting effective amount in the blood and/or tissue of a compound of formula (I). The effective level, with regard to the amount of one or more compounds of formula (I) that is effective to therapeutically inhibit abnormal or undesirable serotonin reuptake in the patient, may be chosen, for example, as the blood or tissue level that corresponds to a concentration of one or more compounds of formula (I) effective to reduce or eliminate the symptoms or behaviors associated with psychiatric diseases or disorders, e.g., based on an assay, which is reasonably predictive of clinical efficacy.

Alternatively or additionally, the effective level can be defined, for example, as the amount required in an individual patient that is effective to kill or inhibit the growth (e.g., suppress, retard or decrease the growth rate) of cells associated with a particular proliferative disease or disorder (i.e., diseases associated with abnormal or undesirable cell proliferation) in the patient as taught in copending U.S. Patent Application No. 60/813,079, which is incorporated in its entirety herein by reference.

One skilled in the art can easily determine the appropriate dose, schedule, and method of administration for the exact formulation or composition being used in order to achieve the desired effective level in the patient. One skilled in the art also can readily determine by a direct (e.g., analytical chemistry) and/or indirect (e.g, with clinical chemistry indicators) analysis of appropriate patient samples (e.g., blood and/or tissues), or, in the case of psychiatric diseases or disorders, e.g., by direct or indirect observations of the individual patient's behavior. The effective level may be achieved, for example, by administering one or more compounds in accordance with the method of the present invention in an amount effective to ameliorate undesired symptoms associated with a psychiatric disease or disorder, prevent the manifestation of such symptoms before they occur, slow the progression of a psychiatric disease or disorder, slow the progression of symptoms associated with a psychiatric disease or disorder, reduce the severity of a psychiatric disease or disorder, cure the disease or disorder, improve the survival rate of patients suffering from the disease or disorder, initiate a more rapid recovery from the disease or disorder, and/or prevent (e.g., decrease the likelihood of) the disease from occurring.

The method of the present invention can thus be applied toward the treatment or prevention of a psychiatric disease or disorder (e.g., a psychiatric disease or disorder associated with abnormal serotonin reuptake). Exemplary diseases or disorders that may be treated or prevented in accordance with the present invention include major depressive disorder, anxiety, social anxiety disorder (SAD), generalized anxiety disorder (GAD), obsessive compulsive disorder (OCD), major depressive disorder (MAD), premenstrual dysphoric disorder (PMDD), panic attacks, panic disorder (PD), posttraumatic stress disorder (PTSD), eating disorders, bulimia nervosa, pain, neuralgic pain, post herpetic neuralgia, phobias of various types, and premenstrual dysphoric disorder (PMDD). In one embodiment, the method of the present invention includes treating or preventing generalized anxiety disorder (GAD), social anxiety disorder, obsessive compulsive disorder (OCD), panic disorder (PD), and/or posttraumatic stress disorder (PTSD). In another embodiment, the method of the present invention includes treating or preventing major depressive disorder, obsessive compulsive disorder (OCD), bulimia nervosa, and/or panic disorder (PD). In yet another embodiment, the method of the present invention includes treating or preventing major depressive disorder, obsessive compulsive disorder (OCD), panic disorder (PD), posttraumatic stress disorder (PTSD), premenstrual dysphoric disorder (PMDD), and/or social anxiety disorder.

Other exemplary diseases or disorders that may be treated or prevented in accordance with the present invention include premature ejaculation, arthritis, chronic fatigue, multiple sclerosis, lupus, irritable bowel syndrome (IBS), migraine headache, diabetic neuropathy, fibromyalgia, attention-deficit/hyperactivity disorder (ADHD), autistic spectrum disorders, bipolar depression, attention deficit disorder, chronic pain, neuralgic pain, postherpetic neuralgia, phobias of various types, eating disorders, panic attacks, and neurocardiogenic syncope.

Since cancer patients are highly susceptible to states of depression, anxiety and other mood disorders, the compounds administered in accordance with the method of the present invention, which possess antiproliferative activity as taught in copending U.S. Patent Application No. 60/813,079, as well as antidepressant, anxiolytic, antidementia, and other psychiatric activities, can be used to treat cancer patients with psychiatric co-morbidity. Accordingly, the present invention further provides a method of treating or preventing a disease or disorder treatable by the inhibition of neurotransmitter reuptake in a patient that has cancer, wherein the method includes administering a neurotransmitter reuptake inhibiting effective amount of at least one compound of the formula A-L-B (I) as described herein. In one embodiment, the method of the present invention includes treating or preventing a psychiatric disease or disorder associated with cancer in the patient (e.g., psychiatric co-morbidity in a cancer patient.

In accordance with the method of the present invention, the dose administered to a patient preferably is sufficient to produce an effective level in the patient over a reasonable time frame. It will be appreciated that the amount of active ingredient that can be combined with the carrier materials to produce a single dosage form will vary depending upon the patient treated and the particular mode of administration. One skilled in the art will recognize that the specific dosage level for any particular patient will depend upon a variety of factors including, for example, the activity of the specific compound employed, age, body weight, general health, sex, diet, time of administration, route of administration, rate of excretion, drug combination and severity of the particular psychiatric disease or disorder being treated. The size of the dose will also be determined by the existence, nature, and extent of any adverse side-effects that might accompany the administration of a particular compound. Other factors which affect the specific dosage can include, for example, bioavailability, metabolic profile, the pharmacodynamics associated with the particular compound to be administered in a particular patient, and the like.

One or more compounds administered in accordance with the method of the present invention can be formulated into a pharmaceutical composition, e.g., by combining a therapeutically effective amount of one or more compounds with a pharmaceutically acceptable carrier. Suitable pharmaceutically acceptable carriers can include carriers that are well-known in the art, e.g., pharmaceutical vehicles, adjuvants, excipients, diluents, and the like. Preferably, the pharmaceutically acceptable carrier is selected such that it is chemically inert with respect to the active agent(s). The carrier also is desirably selected such that it has minimal or no detrimental side effects or toxicity under the conditions of use. The choice of a carrier will be determined in part by the particular composition, as well as by the particular mode of administration.

One skilled in the art will appreciate that various routes of administering a drug are available and, although more than on route may be used to administer a particular drug, one particular route may provide a more immediate and more effective reaction than anther route. Furthermore, one skilled in the art will appreciate that the particular pharmaceutical carrier employed will depend, in part, upon the particular compound employed and the chosen route of administration. For instance, the compounds of formula (I) may be administered using conventional administration and dosing regimens which have been approved for known selective serotonin reuptake inhibitors, such as, for example, Prozac®, Zoloft®, and/or Paxil®.

The pharmaceutical composition, which can be administered in accordance with the method of the present invention, may be in a form suitable for oral administration, such as, for example, tablets, troches, aqueous or oily suspensions, dispersible powders or granules, emulsions, hard or soft capsules, syrups, solutions or elixirs. Compositions intended for oral use may be prepared according to any method known in the art for the manufacture of such pharmaceutical compositions, and such compositions can contain one or more agents including, for example, sweetening agents, flavoring agents, coloring agents, and preserving agents in order to provide a pharmaceutically elegant and/or palatable preparation. Tablets can contain the active ingredient in admixture with non-toxic pharmaceutically acceptable excipients which are suitable for the manufacture of tablets. Such excipients can include, for example, inert diluents such as, for example, calcium carbonate, lactose, mannitol, calcium phosphate or sodium phosphate; granulating and disintegrating agents such as, for example, maize starch, corn starch, potato starch, and alginic acid; binding agents such as, for example, starch, gelatine or acacia, lubricating agents such as, for example, stearic acid or talc, and the like. Such excipients can also include microcrystalline cellulose, colloidal silicon dioxide, croscarmellose, and the like. The tablets may also include other excipients, colorants, diluents, buffering agents, moistening agents, preservatives, flavoring agents, and pharmacologically compatible carriers.

Exemplary excipients can include dibasic calcium phosphate dihydrate, hypromellose, magnesium stearate, polyethylene glycols, polysorbate 80, sodium starch glycolate, titanium dioxide, starch, silicone, iron oxide, hydroxypropyl cellulose, microcrystalline cellulose, magnesium stearate, polyethylene glycol, polysorbate 80, sodium starch glycolate, gelatin, synthetic yellow iron oxide, and the like, and combinations thereof. For instance, one or more compounds of formula (I) can be formulated in the same manner as known selective serotonin reuptake inhibitors are formulated, such as, for example, Prozac® (fluoxetine hydrochloride, capsules, tablets and oral solution as marketed by Lilly), Zoloft® (sertraline hydrochloride tablets and oral concentrate as marketed by Pfizer, and Paxil® (paroxetine hydrochloride tablets as marketed by GlaxoSmithKline).

Tablets may be uncoated, or they may be coated by known techniques to delay disintegration and absorption in the gastrointestinal tract and thereby provide a sustained action over a longer period. A time delay material, for example, glyceryl monostearate or glyceryl distearate, alone or with a wax, may also be employed. Formulations for oral use also can be presented as hard gelatin capsules, wherein the active ingredient is mixed with an inert solid diluent, for example calcium carbonate, calcium phosphate or kaolin, or as soft gelatin capsules wherein the active ingredient is mixed with water or an oil medium, for example arachis oil, peanut oil, liquid paraffin or olive oil.

Furthermore, formulations suitable for oral administration may include liquid solutions, which may consist of an effective amount of one or more compounds in accordance with the method of the present invention dissolved or dispersed in one or more diluents, such as, e.g., water, saline, or orange juice; capsules, sachets or tablets, each containing a predetermined amount of the active ingredient, as solids or granules; solutions or suspensions in an aqueous liquid; and oil-in-water emulsions or water-in-oil emulsions. Aqueous suspensions, for example, can contain the active material(s) in admixture with excipients suitable for the manufacture of aqueous suspensions. Such excipients include suspending agents, for example, sodium carboxymethyl cellulose, methylcellulose, hydroxypropylmethylcellulose, sodium alginate, polyvinylpyrrolidone, gum tragacanth and gam acacia. Dispersing or wetting agents may include natural-occurring phosphatides, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol, for example, polyoxyethylene sorbitol monooleate, or condensation products of ethylene oxide with partial esters derived from fatty acids and hexitol anhydrides, for example, polyoxyethylene sorbitan mono-oleate. The aqueous suspensions also can contain one or more preservatives, for example, ethyl or n-propyl p-hydroxy benzoate, one or more coloring agents, one or more flavoring agents and one or more sweetening agents such as, for example, sucrose or saccharin.

Formulations suitable for oral administration also can include lozenges comprising the active ingredient in a flavor, e.g., sucrose and acacia or tragacanth; pastilles comprising the active ingredient in an inert base, such as, e.g., gelatin and glycerin, or sucrose and acacia; and mouthwashes comprising the active ingredient in a suitable liquid carer; as well as creams, emulsions, gels, and the like containing a therapeutically effective amount of the active ingredient(s).

Oily suspensions may be formulated by suspending the active ingredient in a vegetable oil, for example arachis oil, olive oil, sesame oil or coconut oil, or in a mineral oil such as liquid paraffin. The oil suspensions may contain a thickening agent, for example beeswax, hard paraffin or cetyl alcohol. Sweetening agents, such as those set forth above, and flavoring agents may be added to provide a palatable oral preparation. If desired, such compositions may be preserved by the addition of an antioxidant such as, for example, ascorbic acid, or an antimicrobial agent.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, also may be present.

The pharmaceutical composition associated with the method of the present invention also can be in the form of an oil-in-water emulsion. The oily phase can be a vegetable oil, for example, olive oil or arachis oils, or a mineral oil, for example liquid paraffin or mixtures of these. Suitable emulsifying agents may include naturally-occurring gums, for example gum acacia or gum tragacanth, naturally-occurring phosphatides, for example soya bean lecithin, and esters or partial esters derived from fatty acids and hexitol anhydrides, for example sorbitan mono-oleate, and condensation products of the said partial esters and ethylene oxide, for example polyoxyethylene sorbitan mono-oleate. The emulsions also can contain sweetening and flavoring agents.

The pharmaceutical composition can be in the form of a sterile injectable preparation, for example, as a sterile injectable aqueous or oleagenous suspension. Suitable suspensions for parenteral administration can be formulated according to the known art using those suitable dispersing or wetting agents and suspending agents which have been mentioned above. Formulations suitable for parenteral administration also can include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The sterile injectable preparation can be in the form of a solution or a suspension in a non-toxic parenterally-acceptable diluent or solvent, for example, as a solution in water or 1,3-butanediol. Acceptable vehicles and solvents that can be employed include, for example, water, Ringer's solution and isotonic sodium chloride solution. In addition, sterile, fixed oils are conventionally employed as a solvent or suspending medium. For this purpose any suitable bland fixed oil can be employed including synthetic mono- or diglycerides. In addition, fatty acids such as, for example, oleic acid can find use in the preparation of injectables.

The compound(s) or pharmaceutical composition(s) administered in accordance with the method of the present invention also can be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include, for example, cocoa butter and polyethylene glycols.

Formulations suitable for vaginal administration may be presented as pessaries, tampons, creams, gels, pastes, foams, or spray formulas containing, in addition to the active ingredient, such carriers as are known in the art to be appropriate. Similarly, the active ingredient may be combined with a lubricant as a coating on a condom.

Formulations suitable for topical administration may be presented as creams, gels, pastes, or foams, containing, in addition to the active ingredient, such carriers as are known in the art to be appropriate.

The compound(s) or pharmaceutical composition(s) administered in accordance with the method of the present invention also can be made into aerosol formulations to be administered via inhalation. These aerosol formulations can be placed into pressurized acceptable propellants, such as dichlorodifluoromethane, propane, nitrogen, and the like. They also can be formulated as pharmaceuticals for non-pressured delivery devices such as, e.g., a nebulizer or an atomizer.

The formulations can be presented in unit-dose or multi-dose sealed containers, such as ampules and vials, and can be stored in a freeze-dried (lyophilized) condition requiring only the addition of the sterile liquid excipient, for example, water for injection, immediately prior to use. Extemporaneous injection solutions and suspensions can be prepared from suitable sterile powders or granules.

The compounds administered in accordance with the method of the present invention can be prepared by any suitable process. An exemplary process for preparing such compounds includes reacting a compound of the formula:

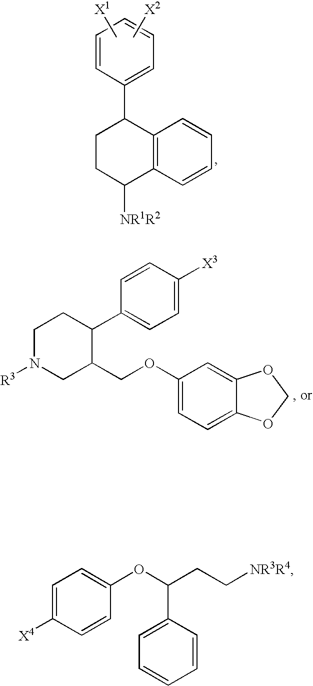

with a halogenating agent to produce a halogenated compound of the formula:

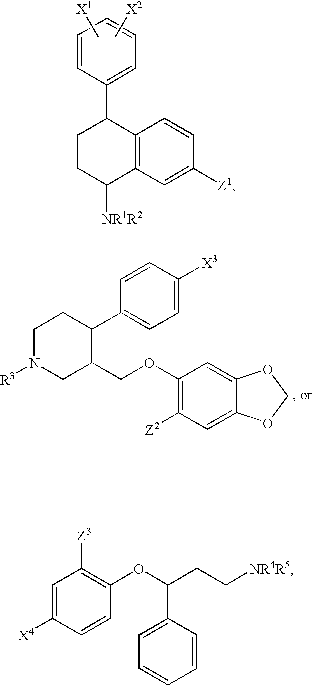

wherein, Z¹, Z², and Z³ are the same or different and each is independently a halogen, and R¹-R⁵ and X¹-X⁴ are as defined herein; coupling the halogenated compound with a compound of the formula L-B, wherein L is a linking group comprising a carbon-carbon triple bond, which is preferably a terminal acetylene (HC≡C—), and B is as defined herein (e.g., alkyl, alkenyl, alkynyl, aralkyl, —(CH₂)ₙ(CHR⁹)ₘQ, —Ar(CH₂)ₙ(CHR⁹)ₘQ, —(CH₂)ₙAr(CHR⁹)ₘQ or —(CH₂)ₙ(CHR⁹)ₘArQ), wherein R¹-R⁹, X¹-X⁴, Q, m and n are as defined herein, to produce a coupling product that includes a carbon-carbon triple bond; optionally converting the carbon-carbon triple bond in the coupling product into a carbon-carbon double bond or carbon-carbon single bond; optionally introducing a pharmaceutically acceptable solubility modifying group to the coupling product; and optionally converting the coupling product into a pharmaceutically acceptable salt, ester, or prodrug, to produce a compound of the formula A-L-B (I) as defined herein.

The halogenating agent can generally include any compound, reagent or combination of compounds and reagents, which is capable of halogenating (preferably by selectively introducing a halogen) to an aromatic ring. Exemplary halogenating agents may include, e.g., Br₂ (with or without a catalyst), Cl₂ (with or without a catalyst), I₂ (with or without a catalyst), N-chlorosuccinimide, N-bromosuccinimide, N-iodosuccinimide, and the like. Preferably, the halogenating agent used in the production process of the present invention is N-chlorosuccinimide, N-bromosuccinimide, or N-iodosuccinimide.

The coupling reaction generally includes methods known in the art for introducing an alkyne substituent to a suitably reactive aromatic halide to produce the coupling product. For instance, the coupling process may be performed by way of a Sonogashira coupling reaction.

Methods for optionally converting the carbon-carbon triple bond into a carbon-carbon double bond or a carbon-carbon single bond are generally known in the art. For example, the carbon-carbon triple bond can be converted into a carbon-carbon double bond or a carbon-carbon single bond via reduction, (e.g., hydrogenation), hydroboration (and, optionally, further reacting the hydroborated intermediate, e.g., by oxidation), hydrohalogenation, halogenation, and the like.

Likewise, methods for optionally introducing a pharmaceutically acceptable solubility modifying group are known in the art. For example, when R⁷ is hydrogen, a pharmaceutically acceptable solubility modifying group may be introduced by esterifying a hydroxyl group with an acylating agent that includes a pharmaceutically acceptable solubility modifying group, or by alkylating a hydroxyl group with a pharmaceutically acceptable solubility modifying group.

In one embodiment, the process of preparing the compounds administered in accordance with the method of the present invention utilizes the following compound as a starting material.

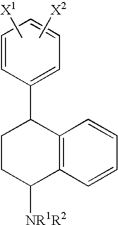

Such starting materials can be obtained using methods, which are well known in the art. See, e.g., U.S. Pat. No. 4,536,518, which describes methods of preparing sertraline and derivatives thereof. An exemplary process is depicted in Scheme 1.

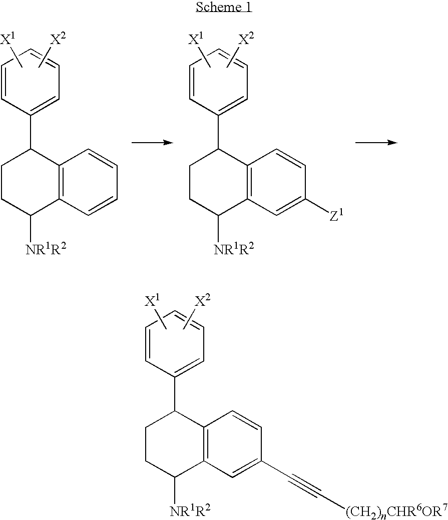

Another exemplary process for preparing the compounds associated with the method of the present invention is depicted in Scheme 2.

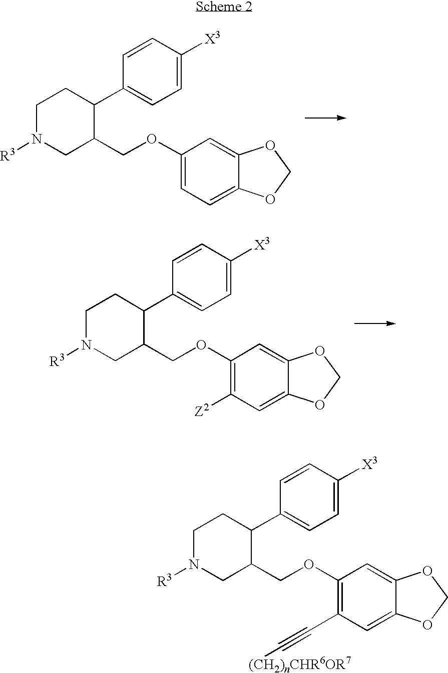

Starting materials for the process depicted in Scheme 2 can be obtained by methods, which are well known in the art. See, e.g., U.S. Pat. No. 4,007,196, which describes methods of preparing paroxetine and derivatives thereof. See also, e.g., U.S. Pat. No. 4,314,081, which describes methods of preparing fluoxetine and derivatives thereof, which can serve as intermediates for producing compounds of the formula A-L-B (I), wherein A is of the formula (A3), as defined herein.

Methods of preparing SSRI derivatives, which can serve as intermediates for producing compounds associated with the method of the present invention, also are described in U.S. Pat. No. 5,320,825.

Another exemplary process for preparing the compounds of formula (I) includes regioselectively formylating a compound of the formula:

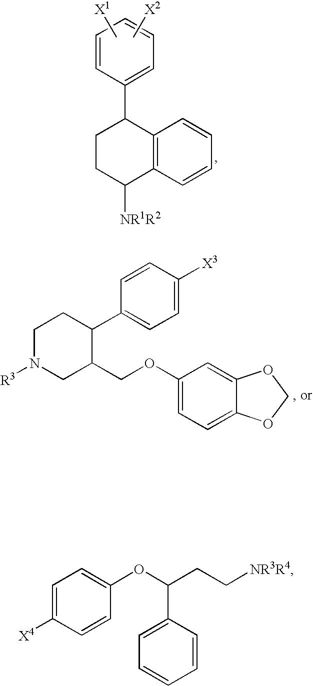

by reacting the compound with a formylating reagent (e.g., using suitable Vilsmeier-Haack conditions), to produce a formylated compound of the formula:

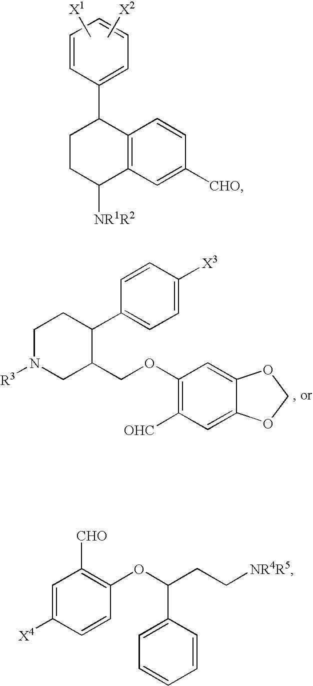

and reacting the formylated compound with a reagent capable of reacting with the formyl substituent (e.g., using a suitable a Wittig reagent or other aldehyde alkenylation reagent), to produce an alkenyl product of the formula:

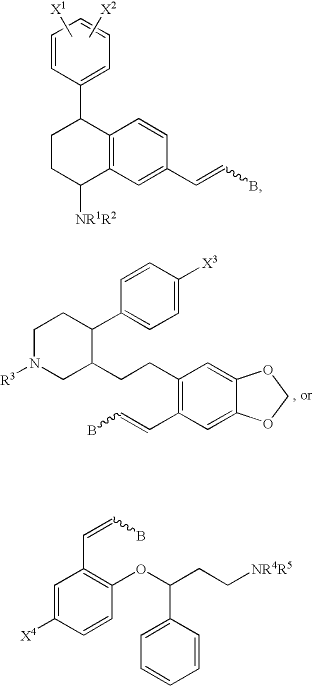

wherein B is as defined herein, and optionally converting the carbon-carbon double bond of the alkenyl product into a carbon-carbon single bond, optionally introducing a pharmaceutically acceptable solubility modifying group to the alkenyl product, and optionally converting the alkenyl product into a pharmaceutically acceptable salt, ester, or prodrug, to produce a compound of the formula A-L-B (I) as defined herein.

The formylating reagent can generally include any compound, reagent or combination of compounds and reagents, which is capable of formylating an aromatic ring. An exemplary formylating reagent, which can be used in the production process of the compounds associated with the method of the present invention, is the product of dimethyl formamide and POCl₃.

The reagent capable of reacting with the formyl substituent can include any compound, reagent or combination of compounds and reagents, which is capable of reacting with the formyl substituent. Exemplary reagents capable of reacting with the formyl substituent may include, e.g., Wittig reagents such as, for example, Ph₃P⁺CH₂B Br⁻, e.g., in the presence of a suitable base, wherein B is as defined herein.

Methods of preparing and using compounds administered in accordance with the method of the present invention (and the antiproliferative activity associated therewith) are disclosed in U.S. Pat. Application No. 60/813,079 filed Jun. 13, 2006, which is incorporated herein by reference.

The compounds associated with the method of the present invention have been assessed using a variety of animal models for anxiety and depression, including open field, elevated plus maze (EPM), forced swim test (FST),

The open field test is now one of the most popular procedure in animal psychology (Prut et al., The open field as a paradigm to measure the effect of drugs on anxiety like behavior: a review, Eur. J. Pharmacol, 463, 3-33 (2003)). The procedure generally involves forced confrontation of a rodent with a situation. The animal is placed in the center or close to the walls of an apparatus and a number of behavioral items are recorded for a period ranging from 5 to 20 min. These behavioral items include horizontal locomotion, frequency of rearing or leaning (sometimes termed vertical activity), and grooming (protracted washing of the coat). When placed in such a situation, rodents spontaneously prefer the periphery of the apparatus to activity in the central parts of the open field. Increases in time spent in the central part, as well as the ratio of central/total locomotion or decrease of the latency to enter the central part, are indications of anxiolysis.

The elevated plus maze is a widely used method to test anxiety in rodents (Pellow et al., Pharmacol. Biochem. Behay., 24, 525-529 (1986)). The apparatus is made of wood and painted black, with two opposing open arms and two opposite enclosed arms of the same size. The arms are attached to a central square shaped in a plus sign. The whole apparatus is placed 50 cm above the floor. Anxious animals refrain from entry to the open arm and prefer the closed arm. Benzodiazepines were shown to increase the time spent in the open arms and the frequency of entries to the open arms (Pellow et al., Validation of open:closed arm entries in an elevated plus-maze as a measure of anxiety in the rat, J. Neurosci. Methods, 14, 149-167 (1985)).

The forced swim test is one of the most widely used tools for screening antidepressant activity preclinically in acute treatment. The test was first described by Porsolt et al. (Porsolt et al., Arch. Int. Pharmacodyn. Ther., 229, 327-336 (1977)). The test is based on the observation that rats and mice develop an immobile posture when placed in an inescapable cylinder of water. This behavior is considered to be a behavioral despair as opposed to an active form of coping with stressful conditions. Three parameters were defined in the evaluation of FST as employed herein. These are immobility, swimming behavior, and climbing behavior. Immobility, is defined in the traditional Porsolt test as when no additional activity is observed other than that required to keep the animal's head above the water. Swimming behavior is defined as the movement (usually horizontal) throughout the chamber that includes crossing into another quadrant. Climbing behavior is defined as upward-directed movements of the forepaws along the side of the swim chamber.

FST is considered a good screen tool with good reliability and predictive validity. However mice and rats of different strains have differential sensitivity to antidepressants from different classes, (e.g., SSRIs, TCAs, SNRIs, etc) and therefore modification of the test were developed in order to have valid and predictive results. The test can be performed acutely or after chronic drug administration in mice and rats. The rats are exposed twice to the antidepressant, while mice develop the immobile posture 1 hr after exposure to the drug.

In mice there are several factors that affect the efficacy, including cylinder diameter, depth of water, interval of scoring, time between treatment and FST, water temperature, and strain of animals and age.

Previous data showed that mice (ICR) strain showed high variability under basal conditions, and were sensitive to TCAs but not to SSRIs, while BalbC strain mice, were found to be one of 3 strains out of 11 sensitive to SSRIs (Lucid et al., Psychopharmacology (Berl)., 155, 315-22 (2001)).

The hot-plate test is widely used to measure pain sensation. The latency in response to heat of mice and rats feet is based on the method described by Eddy et al., Synthetic analgesics. II. Dithienylbutylamines., J. Pharmacol. Exp. Ther. 107(3), 385-393 (1953). The method is based on placing an animal on heated (52-56 C) surface and measuring the time to response (raising paw, licking of the paw, jumping or running.

The following examples further illustrate the invention but, of course, should not be construed as in any way limiting its scope.

### Example 1

This example demonstrates the synthesis of an exemplary compound that can be administered in accordance with the method of the present invention, (1S-cis)-4-(3,4-dichlorophenyl)-7-(5-hydroxy-1-pentyn-1-yl)-1,2,3,4-tetrahydro-N-methyl-1-naphthalenamine hydroiodide (Ia).

(1S-cis)-4-(3,4-dichlorophenyl)-7-iodo-1,2,3,4-tetrahydro-N-methyl-1-naphthalenamine (1) was synthesized, as follows.

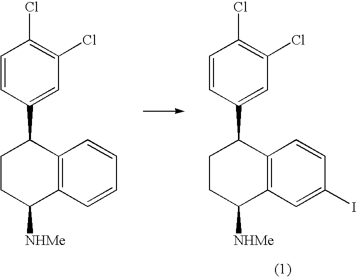

Trifluoromethanesulfonic acid (2.2 ml, 22 mmol) was added to a suspension of (1S-cis)-4-(3,4-dichlorophenyl)-1,2,3,4-tetrahydro-N-methyl-1-naphthalenamine hydrochloride (Sertraline hydrochloride) (2.5 g, 7.3 mmol) in 8 ml dichloromethane (DCM) and cooled to 0° C. under nitrogen. Following the complete dissolution of the salt, N-iodosuccinimide (1.3 g, 6.5 mmol) was added. The reaction was stirred for 17 h and a second portion of N-iodosuccinimide (0.3g, 1.5 mmol) was added. After 24 h, a 2 N aqueous sodium hydroxide solution (15 mL) was added slowly and the resulting mixture was extracted three times with 15 mL of diethyl ether. The combined organic extracts were washed with a saturated aqueous solution of sodium thiosulfate (15 mL) and then brine (15 mL), and dried over MgSO₄. The ether was evaporated under reduced pressure and the crude yellow oil was purified on silica gel (eluent:ethyl acetate:hexanes, 0:100 to 50:50) to yield 1.0 g (35%) of the compound represented by formula (1) as a dark yellow oil.

(1S-cis)-4-(3,4-dichlorophenyl)-7-(5-hydroxy-1-pentyn-1-yl)-1,2,3,4-tetrahydro-N-methyl-1-naphthalenamine hydroiodide (Ia) was synthesized, as follows.

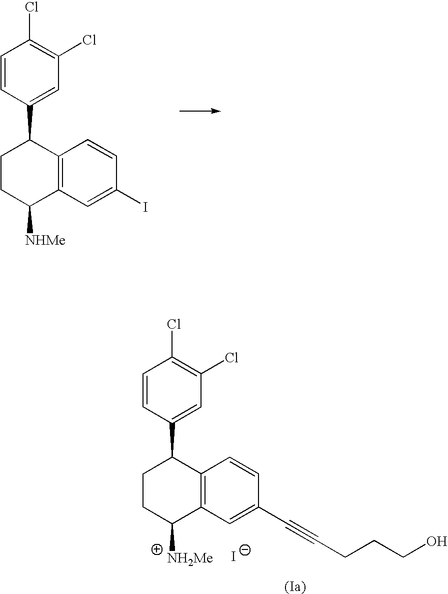

The compound represented by formula (1) (250 mg, 0.58 mmol), Pd(PPh₃)₂Cl₂ (9 mg, 0.013 mmol), CuI (5 mg, 0.025 mmol), 4-pentyn-1-ol (50 mg, 0.64 mmol), diethylamine (0.97 ml), and DMF (1.5 ml) were mixed and stirred under nitrogen overnight. The solvent was removed under reduced pressure and the residue was purified on silica gel (hexanes:ethyl acetate, 50:50 to 0:100) to yield the compound represented by formula (Ia) (70%) as an off-white solid. ¹H NMR (400 MHz, CDCl₃/CD₃OD (2%)): δ 7.50 (br s, 1H), 7.21 (d, J=8.0 Hz, 1H), 7.14 (d, J=1.8 Hz, 1H), 7.06 (dd, J=8.0 Hz, J=1.3 Hz, 1H), 6.93 (dd, J=8.0 Hz, J=2.0 Hz, 1H), 6.63(d, J=8.0 Hz, 1H), 4.21 (br t, J=4.0 Hz, 1H), 3.84 (br t, J=6.8 Hz, 1H), 3.59 (t, J=6.4 Hz, 2H), 2.56 (s, 3H), 2.32 (t, J=6.8 Hz, 2H), 2.09 (m, 1H), 1.94 (m, 3H), 1.65 (quint, J=6.6 Hz; 2H). ¹³C NMR (100 MHz, CDCl₃/CD₃OD (2%)): δ 144.9, 138.0, 133.0, 132.3, 131.8, 131.4, 130.7, 130.6, 130.5, 130.2, 128.5, 122.8, 90.8, 80.0, 61.0, 56.4, 44.6, 31.1, 30.8, 27.7, 23.3, 15.7. HRMS (EI): calcd. for C₂₂H₂₃Cl₂NO (M⁺) 387.1157; found 387.1167.

### Example 2

This example illustrates the synthesis of an exemplary compound that can be administered in accordance with the method of the present invention, (3S-trans)-3-((6-(5-hydroxy-1-pentyn-1-yl)-1,3-benzodioxol-5-yloxy)methyl)-4-(4-fluorophenyl)-piperidine (Ib) was synthesized as outlined in Scheme 3.

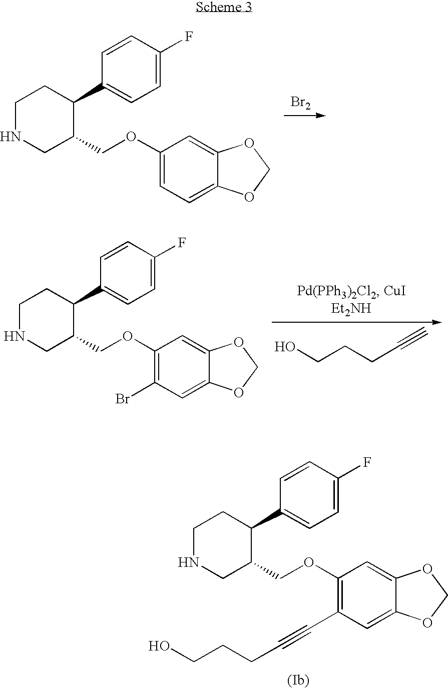

(3S-trans)-3-((6-bromo-1,3-benzodioxol-5-yloxy)methyl)-4-(4-fluorophenyl)-piperidine was synthesized, as follows. A solution of bromine (0.07 mL, 1.44 mmol) in 0.5 mL of dichloromethane was added dropwise to a suspension of (3S-trans)-3-((1,3-benzodioxol-5-yloxy)methyl)-4-(4-fluorophenyl)-piperidine hydrochloride (Paroxetine hydrochloride) (0.5 g, 1.37 mmol) in 4 mL of dichloromethane. After 2 h, 30 mL of water was added and the mixture was extracted with 20 mL of dichloromethane. The organic phase was washed twice with a saturated aqueous solution of sodium bicarbonate (2×30 mL), then with brine (30 mL) and finally with 30 mL of a saturated aqueous solution of sodium sulfite. The dichloromethane solution was dried over MgSO₄, and the solvent was evaporated under reduced pressure. The resulting crude brown oil was purified on silica gel (eluent methanol:ethyl acetate, 0:100 to 40:60) to yield 219 mg (39%) of yellow oil. ¹H NMR (200 MHz, CDCl₃): δ 7.22 (dd, J=8.7 Hz, J=5.5 Hz, 2H), 7.00 (t, J=8.7 Hz, 2H), 6.98 (s, 1H), 6.26 (s, 1H), 5.91 (s, 2H), 3.65 (dd, J=9.2 Hz, J=2.6 Hz, 1H), 3.49 (m, 2H), 3.28 (dm, J=11.2 Hz, 1H), 2.82 (m, 3H), 2.07 (m, 1H), 1.88 (m, 2H). ¹³C NMR (50 MHz, CDCl₃): δ 161.5 (d, J=243 Hz), 150.1, 147.5, 141.8, 139.4, 128.8 (d, J=7 Hz), 115.4 (d, J=21 Hz), 112.3, 101.9, 101.6, 96.7, 70.2, 49.6, 46.5, 43.7, 42.5, 34.4. MS (EI): calcd. for C₁₉H₁₉BrFNO₃ (M⁺) 407.1; found 407.1.

(3S-trans)-3-((6-(5-hydroxy-1-pentyn-1-yl)-1,3-benzodioxol-5-yloxy)methyl)-4-(4-fluorophenyl)-piperidine (Ib) was synthesized, as follows. (3S-trans)-3-((6-bromo-1,3-benzodioxol-5-yloxy)methyl)-4-(4-fluorophenyl)-piperidine (0.22 g, 0.54 mmol), Pd(PPh₃)₂Cl₂ (0.06 g, 0.08 mmol), CuI (0.01 g, 0.06 mmol), triphenylphosphine (0.014 g, 0.055 mmol), 4-pentyn-1-ol (0.06 mL, 0.6 mmol), triethylamine (1 4 mL, 10 mmol) and 5 mL of dry tetrahydrofuran (THF) were mixed and stirred in a pressure tube at 100° C. under nitrogen. After 3 days the solvent was removed under reduced pressure and the crude oil was dissolved in 15 mL of ethyl acetate and washed two times with a saturated aqueous solution of potassium carbonate and then with brine (15 mL each). The organic phase was dried over MgSO₄ and the solvent was evaporated under reduced pressure. The resulting crude brown oil was purified on silica gel (methanol:chloroform, gradient 0:100 to 20:80) to yield 10 mg (5%) of brownish oil. ¹H NMR (400 MHz, CDCl₃): δ 7.18 (dd, J=8.6 Hz, J=5.4 Hz, 2H), 6.99 (t, J=8.7 Hz, 2H), 6.74 (s, 1H), 6.19 (s, 1H), 5.87 (m, 2H), 3.97 (dt, J=10.6 Hz, J=6.8 Hz, 1H), 3.82 (dt, J=10.6 Hz, J=6.8 Hz, 1H), 3.71 (dd, J=12.0 Hz, J=2.8 Hz, 1H), 3.60 (dd, J=9.2 Hz, J=3.0 Hz, 1H), 3.48 (t, J=8.8 Hz, 1H), 3.23 (dm, J=12.1 Hz, 1H), 2.78 (dt, J=3.3 Hz, J=11.9 Hz, 1H), 2.73 (t, J=11.9 Hz, 1H), 2.55 (m, 3H), 2.25 (m, 1H), 1.85 (m, 4H). ¹³C NMR (100 MHz, CDCl₃): δ 161.5 (d, J=243 Hz), 155.6, 147.8, 140.9, 138.7, 128.6, 115.4 (d, J=21 Hz), 111.3, 101.2, 95.9, 92.1, 69.8, 61.1, 49.3, 45.9, 43.7, 42.1, 33.7, 31.5, 15.9. MS (FAB): calcd. for C₂₂H₂₄Cl₂NO (MH⁺) 412.1; found 412.1.

### Example 3

This example illustrates the synthesis of an exemplary compound that can be administered in accordance with the method of the present invention. (1S-cis)-4-(3,4-dichlorophenyl)-7-(5-methoxy-1-pentyn-1-yl)-1,2,3,4-tetrahydro-N-methyl-1-naphthalenamine hydrochloride (Ic) was synthesized as outlined in Scheme 4.

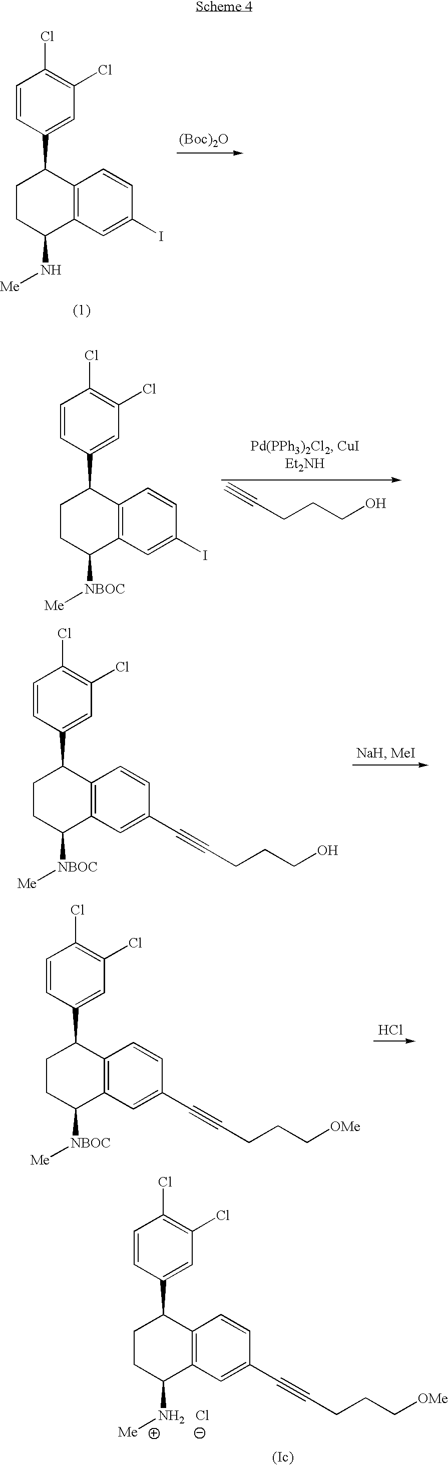

(1S-cis)-4-(3,4-dichlorophenyl)-7-iodo-1,2,3,4-tetrahydro-N-tert-butoxycarbonyl-N-methyl-1-naphtalenamine was synthesized as follows. Di-tert-butyl dicarbonate (0.42 g, 1.9 mmol) was added to a solution of a compound represented by formula (1) (0.76 g, 1.8 mmol) and diisopropylethylamine (0.33 mL, 1.8 mmol) in 20 mL dichloromethane under nitrogen. After 22 h the reaction mixture was washed with aqueous citric acid solution (3×20 mL). The aqueous phase was extracted with 20 mL dichloromethane and the combined organic phase was then washed with brine (20 mL) and dried over Na₂SO₄. Dichloromethane was evaporated to yield 0.69 g (74%) of crude brownish oil, which was used without further purification for the next step. In the NMR spectrum two rotamers are observable (minor rotamer data in square parentheses). ¹H NMR (400 MHz, CDCl₃): δ 7.42 (br s, 1H), 7.38 (d, J=8.0 Hz, 1H), 7.22 (d, J=8.4 Hz, 1H), 6.97 (br s, 1H), 6.68 (br s, 1H), 6.58 (d, J=8.0 Hz, 1H), 5.29 (br s, 1H), [5.12 (br s, 1H)], 4.01 (m, 1H), 2.53 (s, 3H), 2.12 (m, 1H), 1.88 (m, 1H), 1.60 (m, 2H), 1.42 (s, 9H). ¹³C NMR (100 MHz, CDCl₃): δ 156.4, 146.6, 146.3, 139.1, 137.7, 136.2, 136.0, 132.3, 131.9, 130.4, 130.0, 127.8, 92.8, 80.0, 54.7, 53.7, 42.6, 29.8, 28.3, [21.7], 21.2. MS (FAB): calcd. for C₁₇H¹⁷Cl₂IN ((M-BOC)H₂⁺) 432.0; found 431.9.

(1S-cis)-4-(3,4-dichlorophenyl)-7-(5-hydroxy-1-pentyn-1-yl)-1,2,3,4-tetrahydro-N-tert-butoxycarbonyl-N-methyl-1-naphtalenamine was synthesized, as follows. (1 S-cis)-4-(3,4-dichlorophenyl)-7-iodo-1,2,3,4-tetrahydro-N-tert-butoxycarbonyl-N-methyl-1-naphtalenamine (0.69 g, 1.3 mmol), Pd(PPh₃)₂Cl₂ (0.02 g, 0.26 mmol), CuI (0.01 g, 0.055 mmol), 4-pentyn-1-ol (0.13 mL, 1.4 mmol), diethylamine (2.2 mL, 21 mmol) and dry DMF (4 mL) were mixed and stirred under nitrogen overnight. The solvent was removed under reduced pressure and the resulting crude brown oil was dissolved in ethyl acetate (15 mL) and washed with a saturated aqueous solution of potassium carbonate (15 mL) The organic phase was washed with brine (15 mL) and dried over MgSO₄. Ethyl acetate was evaporated under reduced pressure and the resulting crude yellow oil was purified on silica gel (eluent:ethyl acetate:hexanes, 0:100 to 30:70) to yield 0.23 g (36%) of brown oil. In the NMR spectrum two rotamers are observable (minor rotamer data in square parentheses). ¹H NMR (400 MHz, CDCl₃): δ 7.17 (d, J=8.0 Hz, 1H), 7.10 (br m, 1H), 7.05 (d, J=8.0 Hz, 1H), 6.91 (br s, 1H), 6.71 (d, J=8.0 Hz, 1H), 6.66 (br m, 1H), 5.27 (br m, 1H), [5.10 (br m, 1H)], 3.99 (m, 1H), 3.65 (t, J=6.2 Hz, 2H), 2.49 (br s, 3H), 2.39 (t, J=7.0 Hz, 2H), 2.27 (s, 1H), 2.10 (m, 1H), 1.84 (m, 1H), 1.72 (quin, J=6.4 Hz, 2H), 1.58 (m, 2H), 1.38 (s, 9H). ¹³C NMR (100 MHz, CDCl₃): δ 156.3, 146.6, 146.5, 137.8, [137.4], 136.6, 132.2, 130.5, 130.4, 130.3, 130.2, 129.9, 127.8, 122.6, 89.3, 80.5, 79.7, 61.6, 54.9, 53.8, 42.8, 31.2, 29.9, 28.3, [21.9], 21.4, 15.8. MS (FAB): calcd. for C₂₂H₂₄Cl₂NO ((M-BOC)H₂⁺) 388.1; found 388.1.

(1S-cis)-4-(3,4-dichlorophenyl)-7-(5-methoxy-1-pentyn-1-yl)-1,2,3,4-tetrahydro-N-tert-butoxycarbonyl-N-methyl-1-naphtalenamine was synthesized, as follows. A solution of (1S-cis)-4-(3,4-dichlorophenyl)-7-(5-hydroxy-1-pentyn-1-yl)-1,2,3,4-tetrahydro-N-tert-butoxycarbonyl-N-methyl-1-naphtalenamine (0.12 g, 0.24 mmol) in 0.36 mL of dry THF, was added to sodium hydride (0.035 g, 0.72 mmol; 55%-65% in mineral oil) at 0° C. in a pressure tube under nitrogen. The mixture was stirred for 30 min, followed by the addition of methyl iodide (0.05 mL, 0.72 mmol) in 0.24 mL of dry THF. The tube was sealed and, after 5 min, the reaction mixture was allowed to worm to room temperature and then brought to 60° C. After 3 days, excess sodium hydride was destroyed by the dropwise addition of a saturated aqueous solution of ammonium chloride to the cooled (0° C.) reaction mixture and the resulting solution was extracted three times with ethyl acetate (5 mL). The combined organic extracts were washed with a saturated aqueous solution of ammonium chloride and then with brine (15 mL each), and dried over MgSO₄. The ethyl acetate was evaporated under reduced pressure and the crude oil purified on silica gel (eluent:ethyl acetate:hexanes, 0:100 to 5:95) to yield 40 mg (33%) of brown oil. In the NMR spectrum two rotamers are observable (minor rotamer data in square parentheses). ¹H NMR (400 MHz, CDCl₃): δ 7.32 (d, J=8.4 Hz, 1H), 7.24 (br s, 1H), 7.21 (d, J=8.0 Hz, 1H), 7.05 (br s, 1H), 6.87 (d, J=8.0 Hz, 1H), 6.79 (br m, 1H), 5.42 (br m, 1H), [5.24 (br m, 1H)], 4.15 (m, 1H), 3.53 (t, J=6.2 Hz, 2H), 3.37 (s, 3H), 2.62 (br s, 3H), 2.51 (t, J=7.0 Hz, 2H), 2.24 (m, 1H), 1.98 (m, 1H), 1.87 (quin, J=6.6 Hz, 2H), 1.72 (m, 2H), 1.52 (s, 9H). ¹³C NMR (100 MHz, CDCl₃): δ 156.1, 146.6, 146.5, 137.4, [137.1], 136.4, 131.9, 130.2, 130.2, 130.0, 129.9, 129.7, 127.7, 122.7, 89.2, 80.3, 79.4, 70.8, 58.1, 54.2, 53.6, 42.6, 29.7, 28.4, 28.1, [21.7], 21.2, 15.7.

(1S-cis)-4-(3,4-dichlorophenyl)-7-(5-methoxy-1-pentyn-1-yl)-1,2,3,4-tetrahydro-N-methyl-1-naphthalenamine hydrochloride (Ic) was synthesized, as follows. (1S-cis)-4-(3,4-dichlorophenyl)-7-(5-methoxy-1-pentyn-1-yl)-1,2,3,4-tetrahydro-N-tert-butoxycarbonyl-N-methyl-1-naphtalenamine (0.04 g, 0.08 mmol) was dissolved in 2 mL of 4M HCl in dioxan. After 3 h the solvent was removed in vacuo to yield 18 mg (52%) of the compound represented by formula (Ic) as an off-white solid. ¹H NMR (400 MHz, CD₃OD): δ 7.58 (s, 1H), 7.46 (d, J=8.0 Hz, 1H), 7.41 (s, 1H), 7.28 (d, J=8.0 Hz, 1H), 7.18 (br d, J=7.7 Hz, 1H), 6.83 (d, J=8.0 Hz, 1H), 4.45 (br s, 1H), 4.12 (br m, 1H), 3.50 (t, J=6.0 Hz, 2H), 3.32 (s, 3H), 2.81 (s, 3H), 2.46 (t, J=7.0 Hz, 2H), 2.26 (m, 1H), 2.14 (m, 2H), 1.98 (m, 1H), 1.80(quin, J=6.4 Hz, 2H). MS (FAB): calcd. for C₂₃H₂₆Cl₂NO (MH+) 402.1; found 402.1.

### Example 4

This example illustrates a proposed synthesis of an exemplary compound that can be administered in accordance with the method of the present invention. The synthesis is outlined in Scheme 5.

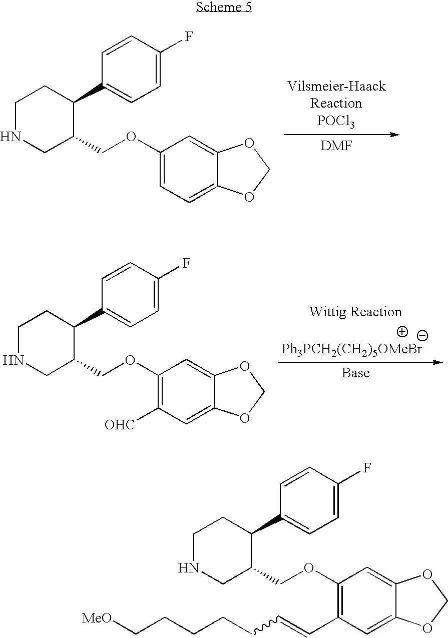

### Example 5

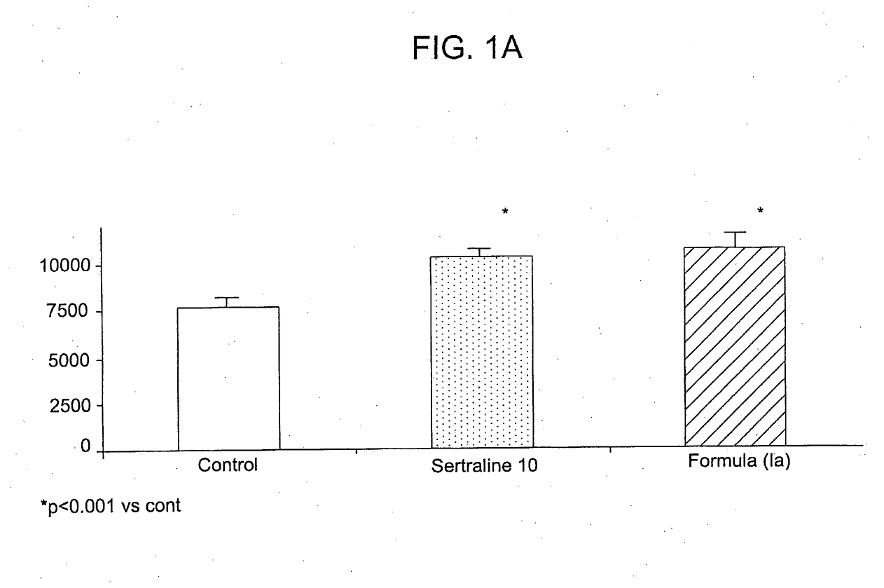

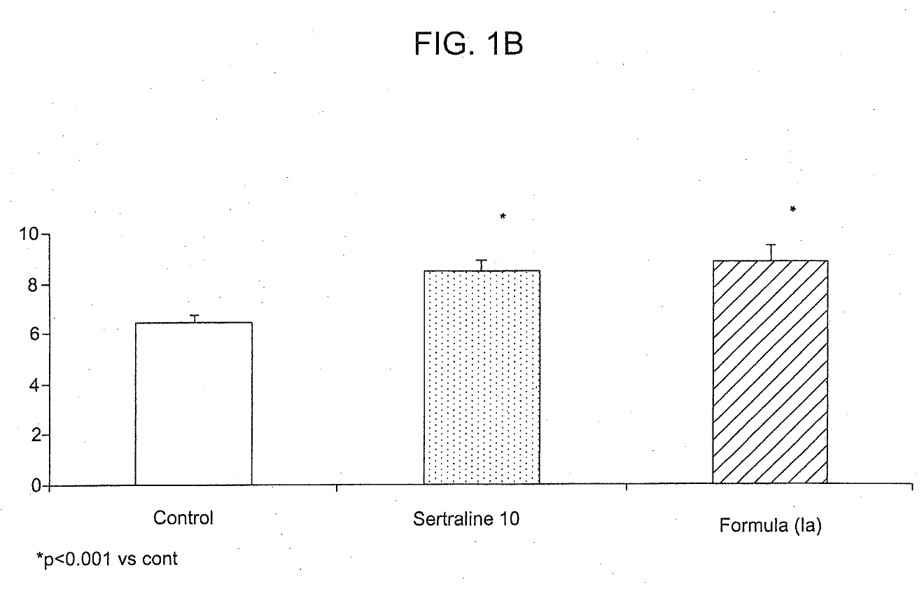

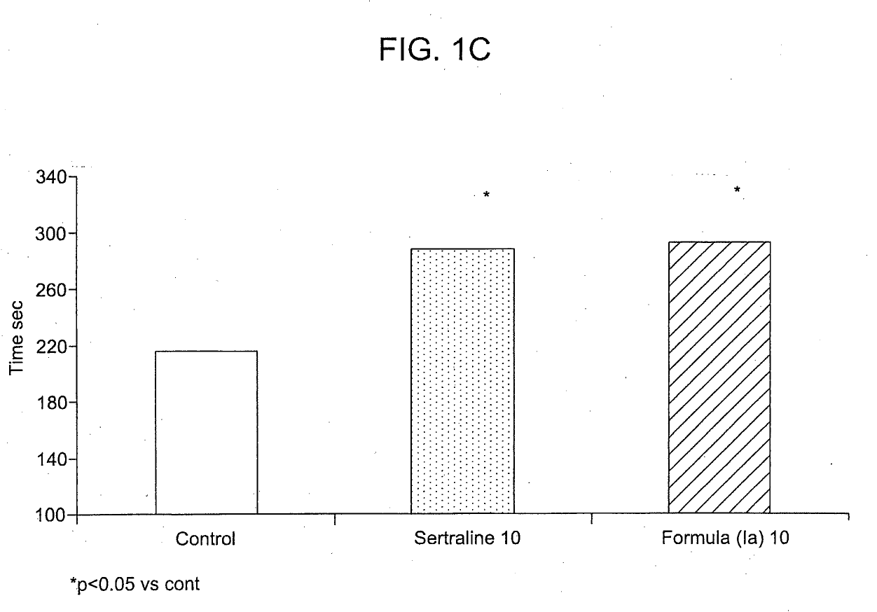

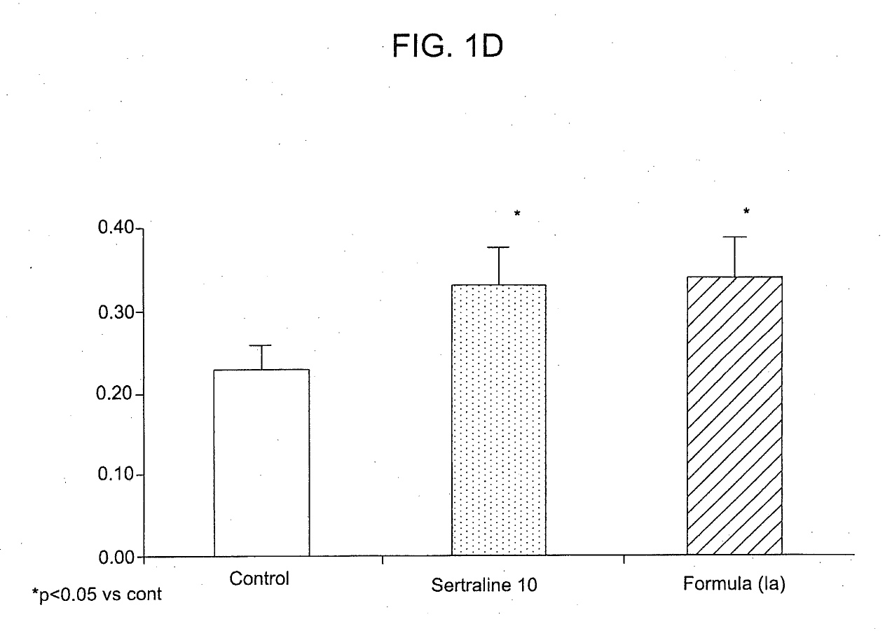

This example demonstrates the effect of an exemplary compound relative to sertraline (control) on distance moved, velocity, total duration in the center, and ratio of total duration between 2+3 to zone 1 in BALB/c mice. FIGS. 1A, 1B, 1C and 1D are bar figures illustrating data for control animals and animals to which equimolar doses (10 mg/kg) of sertraline and the compound of formula (Ia) were administered. The sertraline and the compound of formula (Ia) were administered ip 1 hr prior to placing the animal in the open field. The bar graphs provide data on distance moved, velocity and duration time in the center of the open field. Each column is the mean±SEM of 12 BALB/c mice.

The results demonstrate that both sertraline and the compound of formula (Ia) significantly increase (p<0.001) the velocity and the distance moved of the mice in the exploration field as compared to controls. The results also demonstrate that both sertraline and the compound of formula (Ia) significantly increase the total time spent in the center (areas 2+3) and the ratio (center/periphery), suggesting a similar CNS activity for sertraline and the compound of formula (Ia) and an anxiolytic effect as manifested by more time spent in the center and higher frequency to the center (1.23, 1.27 and 1.35 for controls, sertraline and the compound of formula (Ia) respectively.)

### Example 6

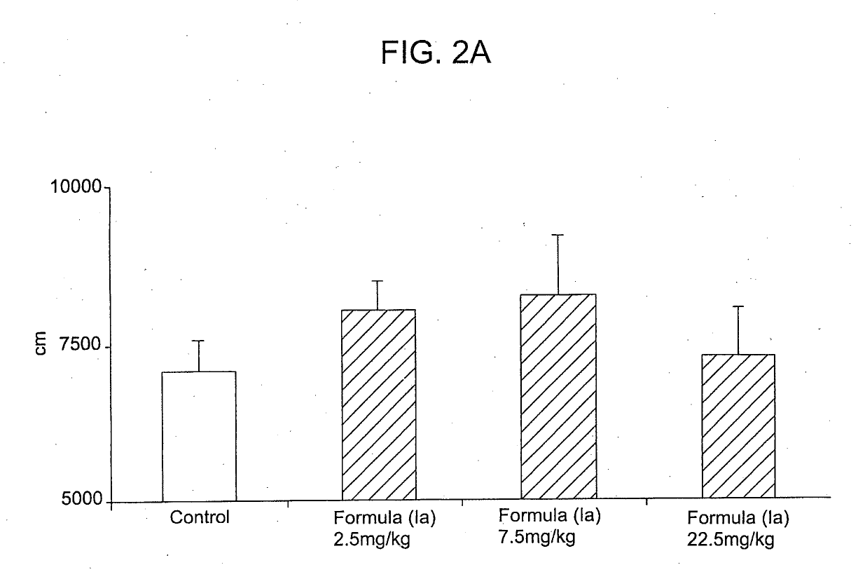

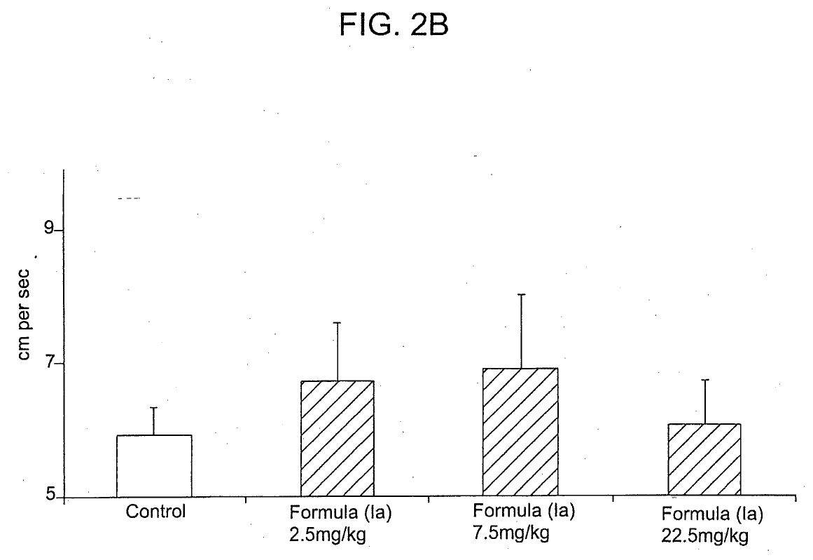

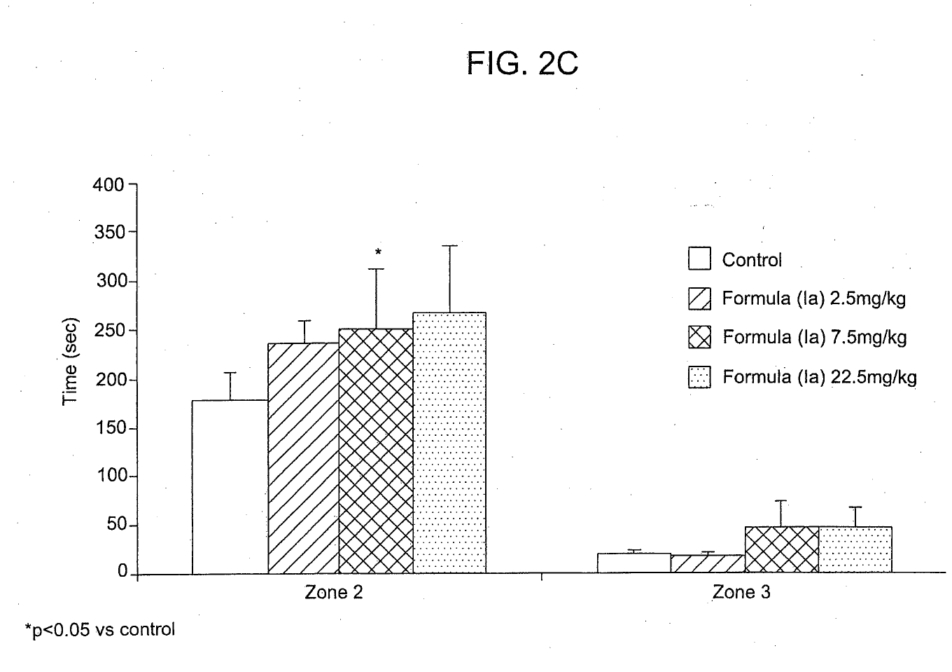

This example demonstrates the effect of an exemplary compound on distance moved, velocity, and total duration in the center in BALB/c mice. FIGS. 2A, 2B, 2C are bar figures illustrate data indicating the presence of a dose dependent effect of the compound of formula (Ia) equimolar to sertraline (2.5,7.5 and 22.5 mg/kg). The compound of formula (Ia) and sertraline were administered to BALB/c mice orally 90 min. prior to placing the animal in the open field. FIGS. 2A, 2B, and 2C provide distance moved, velocity and duration time in the center, respectively. Each column is the mean±SEM of 9 BALB/c mice.

The results show that the compound of formula (Ia) induced a tendency toward slightly increased (2.5 and 7.5 mg/kg) velocity and distance moved, and a dose dependent increase in the time spent in the center (zones 2 and 3) (p<0.05 vs cont).

The data also suggest that the compound of formula (Ia) is rapidly absorbed by oral administration and induces an anxiolytic activity, as reflected by more time spent in the center, and higher frequency of entering the center. The anxiolytic activity is not accompanied by a sedative effect, as reflected by tendency toward increased velocity and distance moved compared to controls.

### Example 7

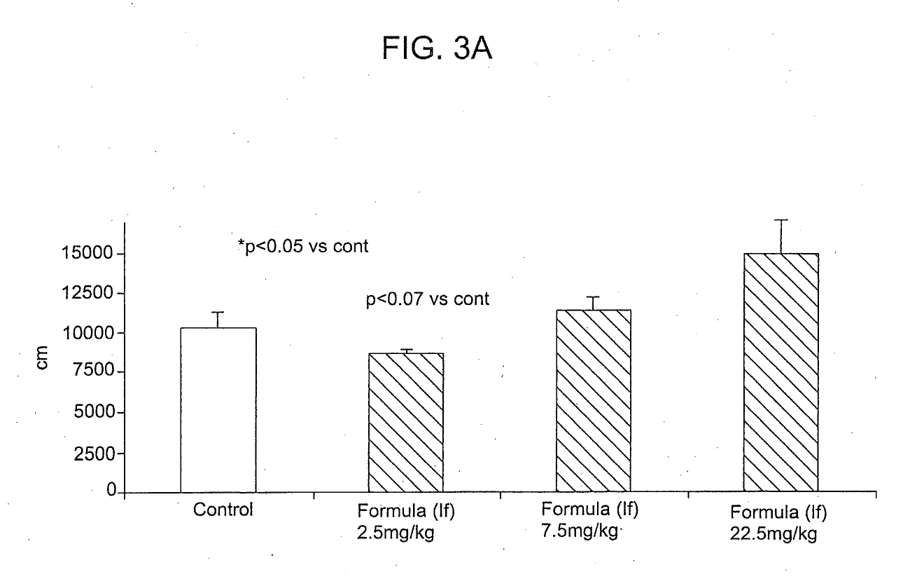

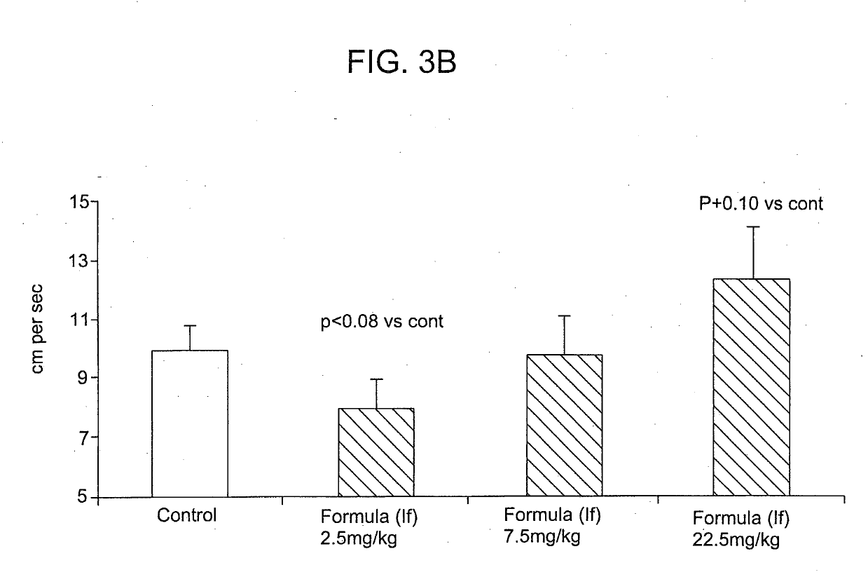

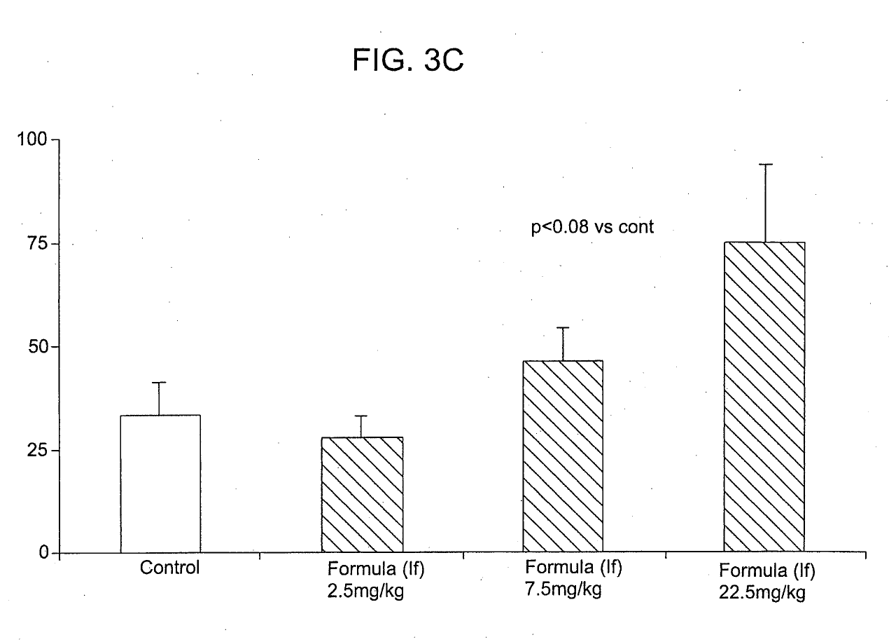

This example demonstrates the effect of an exemplary compound on distance moved, velocity, and frequency to the center in BALB/c mice. FIGS. 3A, 3B, 3C are bar graphs illustrating data indicating the existence of a dose dependent effect for the compound of formula (If) (2.5, 7.5 and 22.5 mg/kg). The compound of formula (If) was administered to BALB/c mice orally 90 min. prior to placing the animal in the open field. These figures provide distance moved, velocity and duration time in the center. Each column is the mean±SEM of 4 animals.

The results indicate that the compound of formula (If) given orally provided increases (7.5 and 22.5 mg/kg) in velocity and distance moved, and a dose dependent increase in the time spent in the center (zones 2 and 3) (p=0.08 vs cont).

The data suggests that the compound of formula (If) is rapidly absorbed by oral administration and induces an anxiolytic activity, as reflected by more time spent in the center and higher frequency of entering the center. Similar to the compound of formula (Ia), the anxiolytic activity (at the high dose) is not accompanied by a sedative effect, as reflected by tendency toward increased velocity and distance moved compared to the controls.

### Example 8

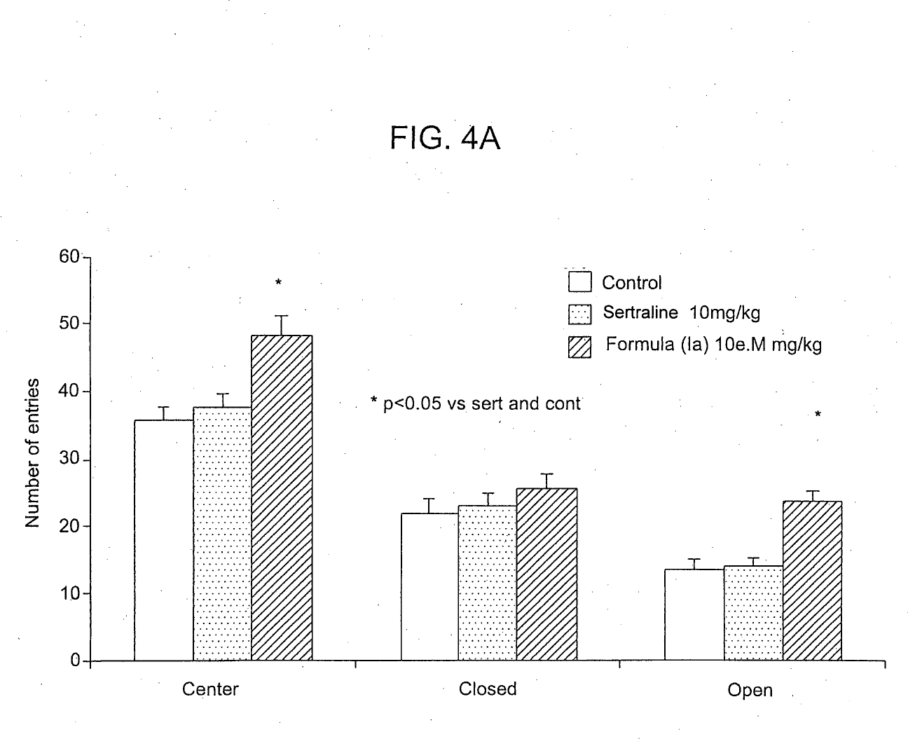

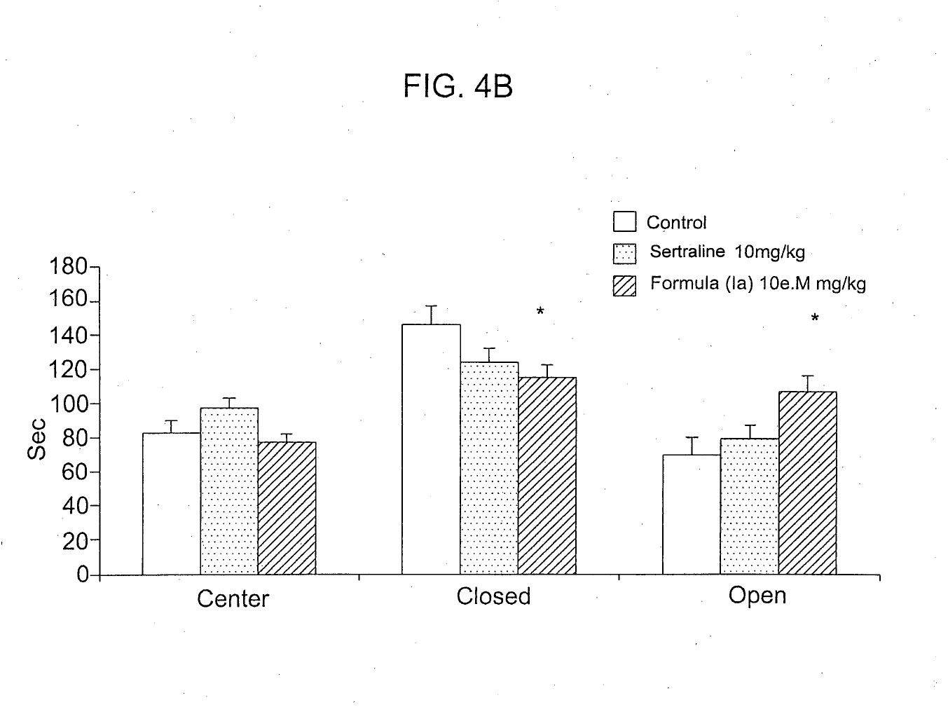

The example demonstrates the effect of an exemplary compound relative to sertraline (control) on frequency to the arms and total duration in the elevated plus maze in BALB/c mice. FIGS. 4A and 4B are bar graphs illustrating data derived from experiments that were performed on the elevated plus maze model using BALB/c mice. Treatment groups consisted of controls (DMSO1%) and animals to which sertraline 10 mg/kg and the compound of formula (Ia) (sertraline equimolar of 10 mg/kg) were administered ip 1 hr prior to maze setting. FIG. 4A provides the number of entries to the different areas and FIG. 4B provides the total duration in the open and the closed arms. The data is presented as the mean±SEM of 12 animals.

The data presented here illustrates that in the elevated plus maze there is a significant anti-anxiety effect produced by the compound of formula (Ia), but not for sertraline at equimolar concentration. These results support a CNS activity for the compound of formula (Ia) and suggest a possible higher or more rapid anxiolytic activity for the compound of formula (Ia), as compared to sertraline.

### Example 9

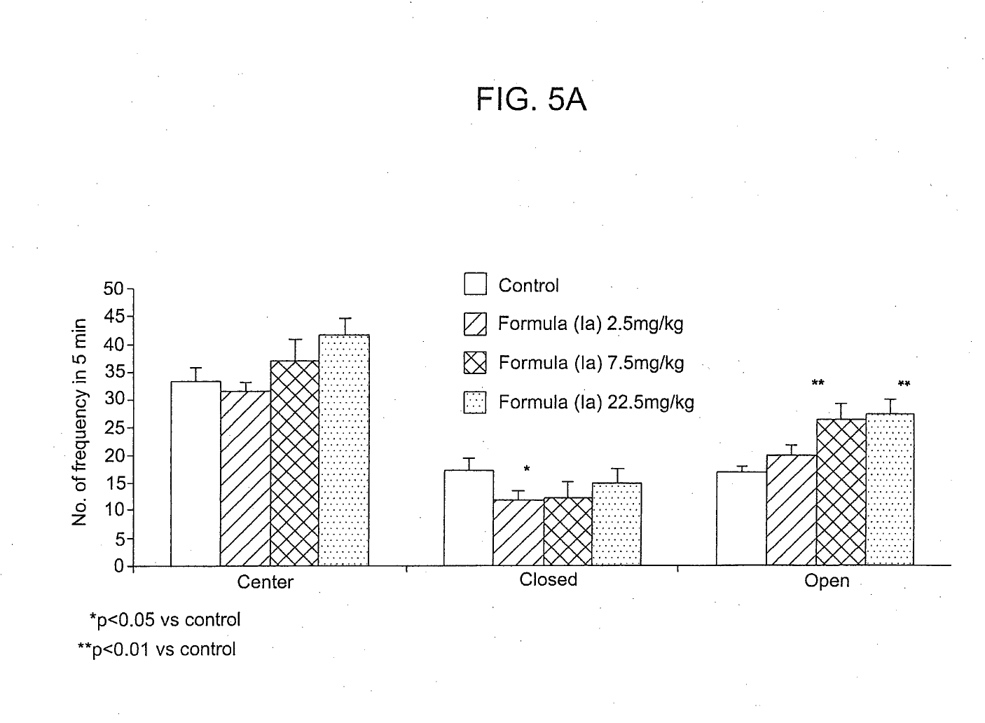

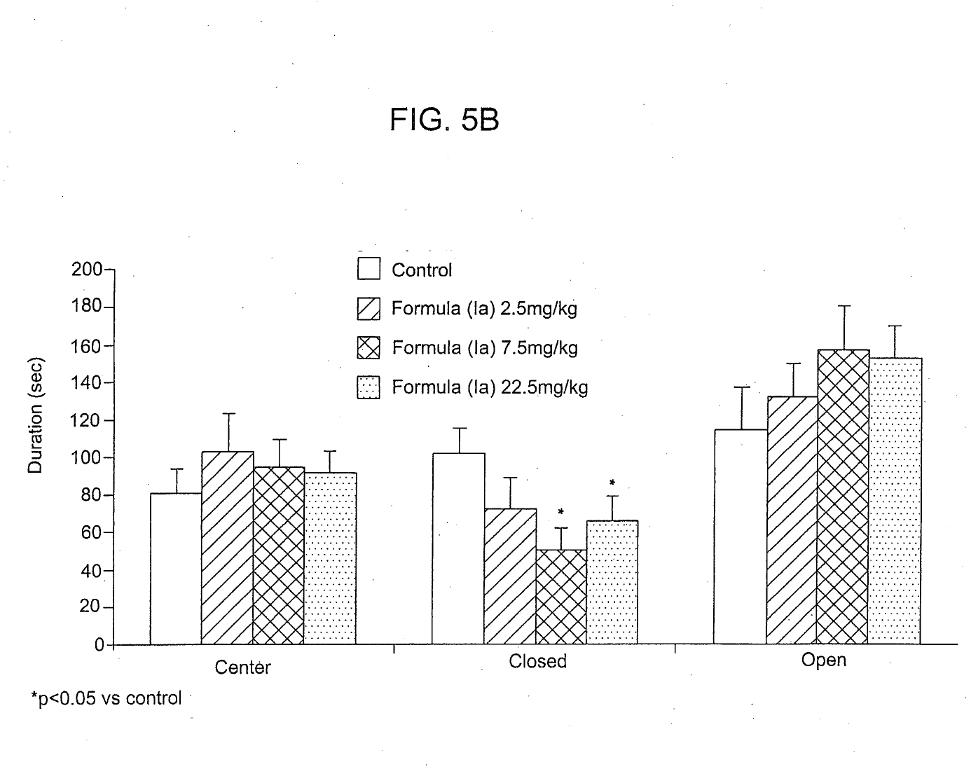

This example demonstrates the effect of an exemplary compound frequency to zone and total duration in the elevated plus maze in BALB/c mice. FIGS. 5A and 5B are bar graphs depicting data for a set of controls (DMSO1%), and for the compound of formula (Ia) (2.5, 7.5 and 22.5 mg/kg). The compound of formula (Ia) and the control were administered to BALB/c mice orally 90 min. prior to elevated plus maze setting. The figures provide the number of entries to the different areas and the total duration in the open and closed arms.

The results indicate a dose dependent anxiolytic effect for oral administration of the compound of formula (Ia). Animals treated with the compound of formula (Ia) spent more time in the open arms and less time in the closed arms. In addition, animals treated with the compound of formula (Ia) frequented the open arm and to the center more often and entered the closed arms less often, compared to controls. These results support the idea that the compound of formula (Ia) might have an acute anxiolytic activity when given by orally.

### Example 10

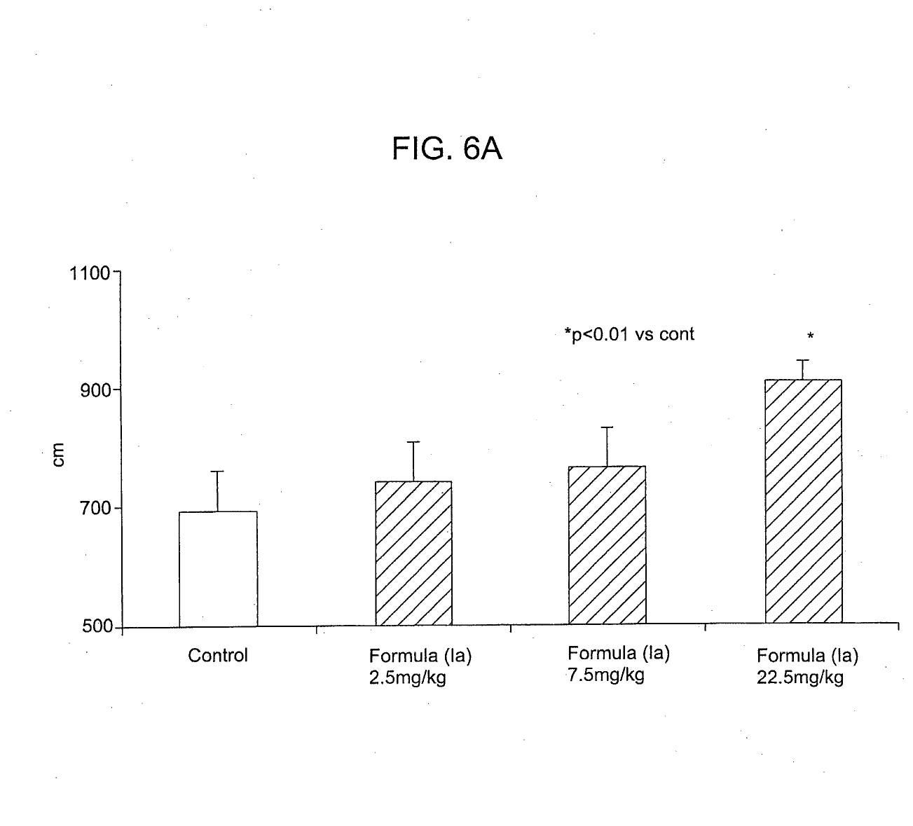

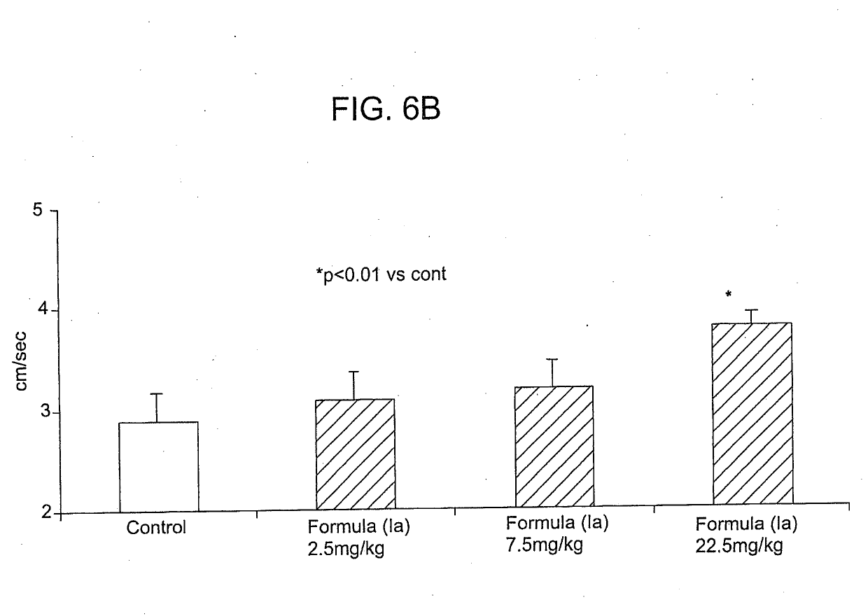

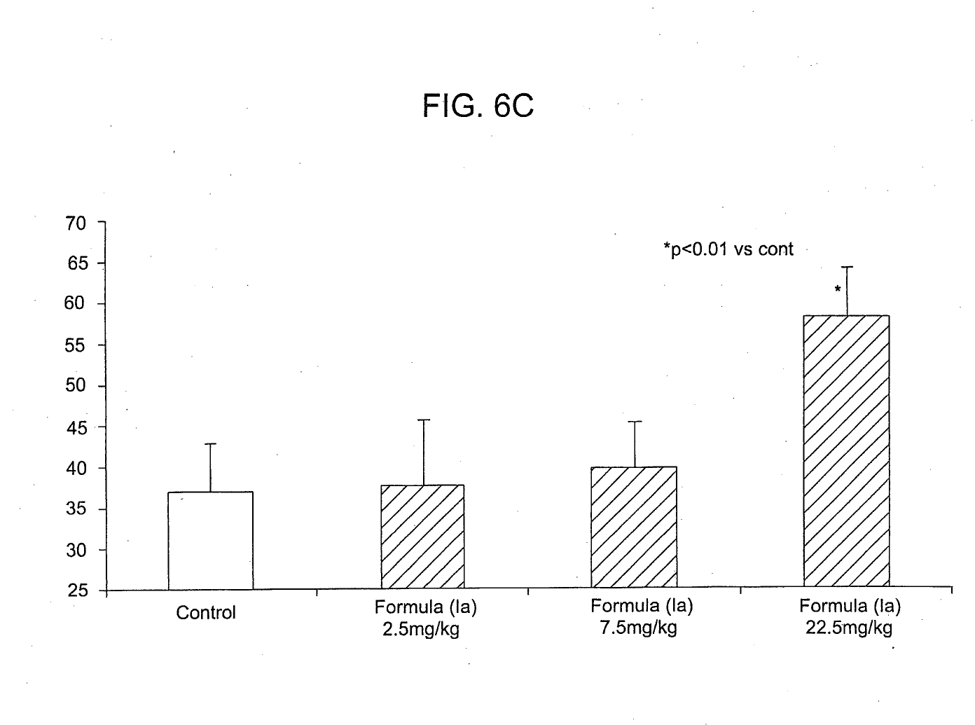

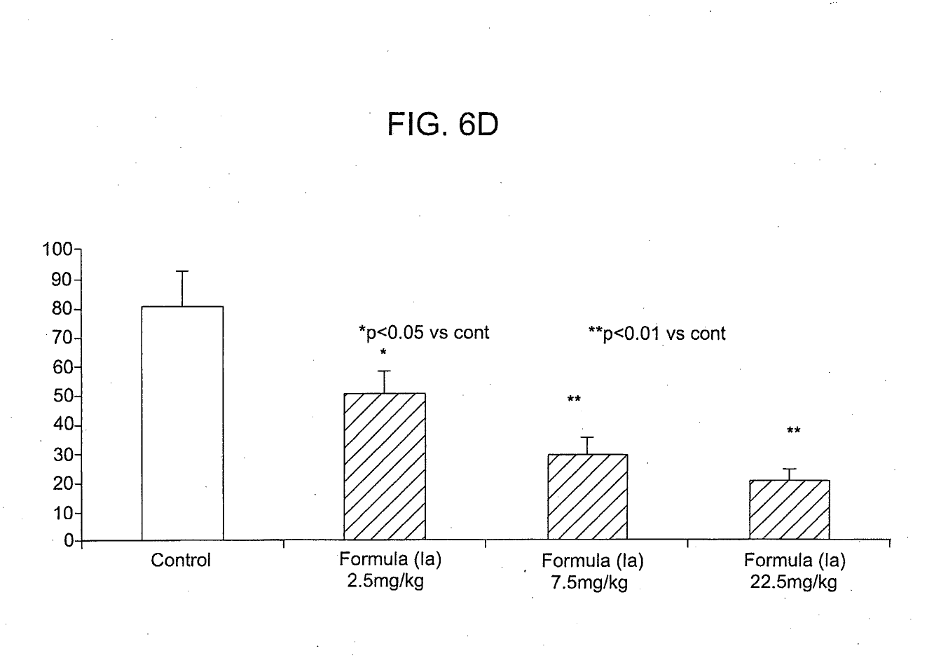

This example demonstrates the effect of an exemplary compound on distance moved, velocity, strong mobility and immobility in the FST in BALB/c mice. FIGS. 6A, 6B, 6C and 6D are bar graphs illustrating data for forced swim tests. The figures illustrate the effect of the compound of formula (Ia) at 2.5, 7.5 and 22.5 mg/kg that was administered ip to BALB/c mice one hr prior to placing the mice in the water. FIGS. 6A and 6B illustrate the effect of the compound of formula (Ia) on distance moved and velocity, respectively, representing the swimming behavior. FIGS. 6C and 6D illustrate the effect of the compound of formula (Ia) on strong mobility, which represents mainly the climbing behavior, and on immobility, respectively. Each bar represents the mean±SEM of 9 animals

The results indicate that the compound of formula (Ia) causes a dose dependent increase in velocity, distance moved and strong mobility, with significant effect reached at 22.5 mg/kg, as compared to control mice. With regard to immobility, a significant dose dependent decrease in immobility was observed with the compound of formula (Ia), which was significant compared to controls, already at the lowest dose of 2.5 mg/kg.

Collectively the data indicate that the compound of formula (Ia) possesses a potent antidepressant effect, which was represented by the motivated swimming activity of the mice.

### Example 11

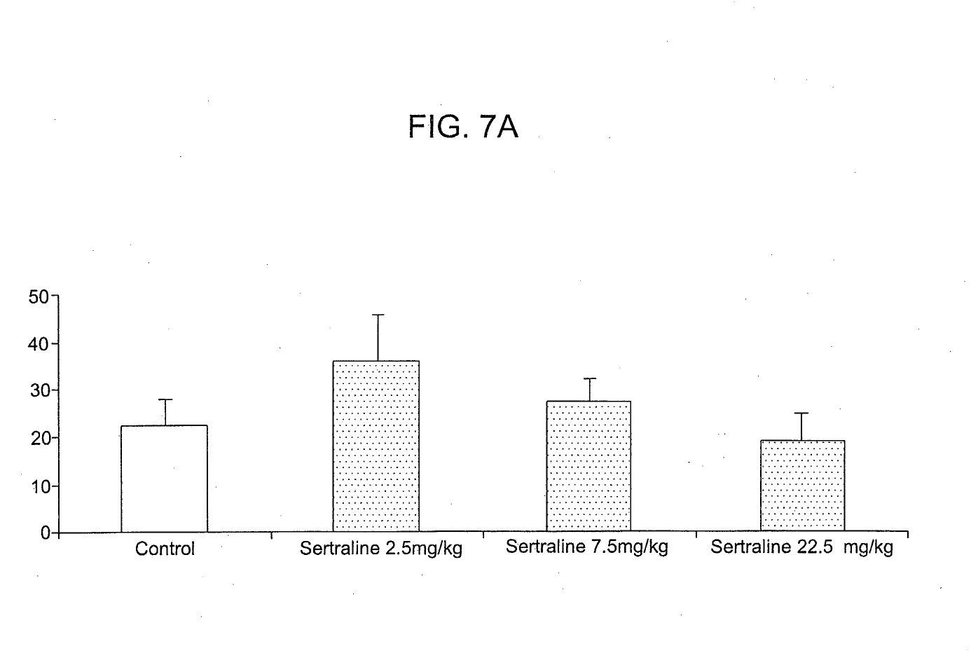

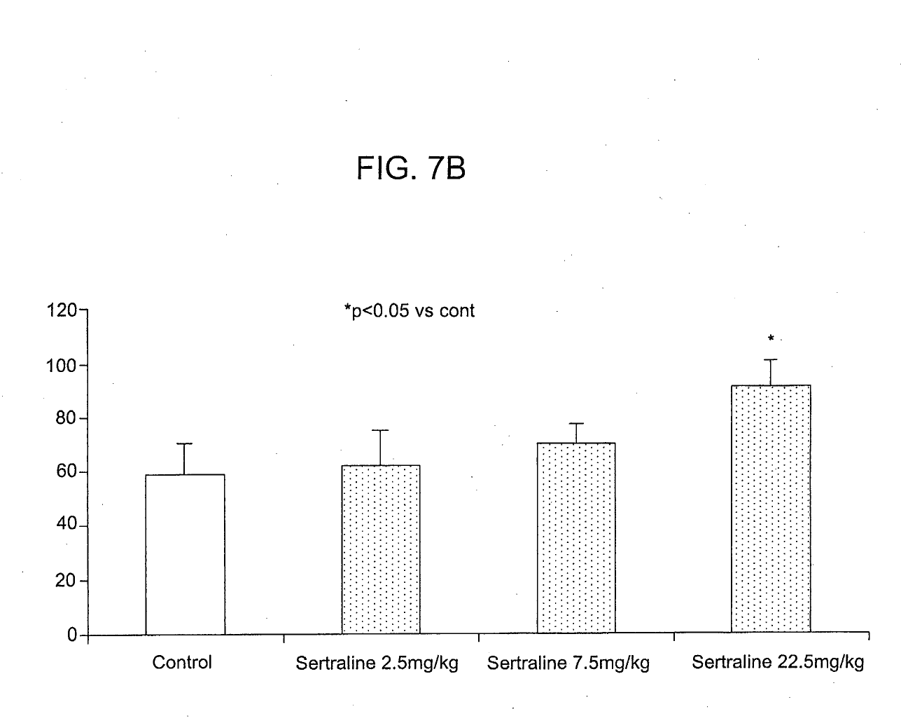

This example demonstrates the effect of an exemplary compound relative to sertraline (control) on strong mobility and immobility in the FST in BALB/c mice. FIGS. 7A and 7B are bar graphs depicting data derived from the administration of sertraline at 2.5, 7.5 and 22.5 mg/kg to BALB/c mice. The sertraline was administered ip one hr prior to placing the mice in the water. The results show the effect of sertraline on strong mobility, which represents mainly the climbing behavior (see FIG. 7A) and the effect on immobility (see FIG. 7B). Each bar represents the mean±SEM of 9 BALB/c mice

The data indicate that sertraline causes a dose dependent decrease in strong mobility and increases in immobility. At the low dose of 2.5 mg/kg a tendency toward increased strong mobility was observed, which did not however reach significance of difference. However, a decrease in immobility was not observed in conjunction with the administration of sertraline.

The data also suggests a differential effect for the compound of formula (Ia) and sertraline. The compound of formula (Ia) seems to be more active in inducing motivated swimming, as compared to sertraline. Moreover, these compounds seems to be different in the dose effect relationship, with sertraline showing an effect at low doses and decrease at higher doses and the compound of formula (Ia) showing a positive relationship between the two factors.

### Example 12

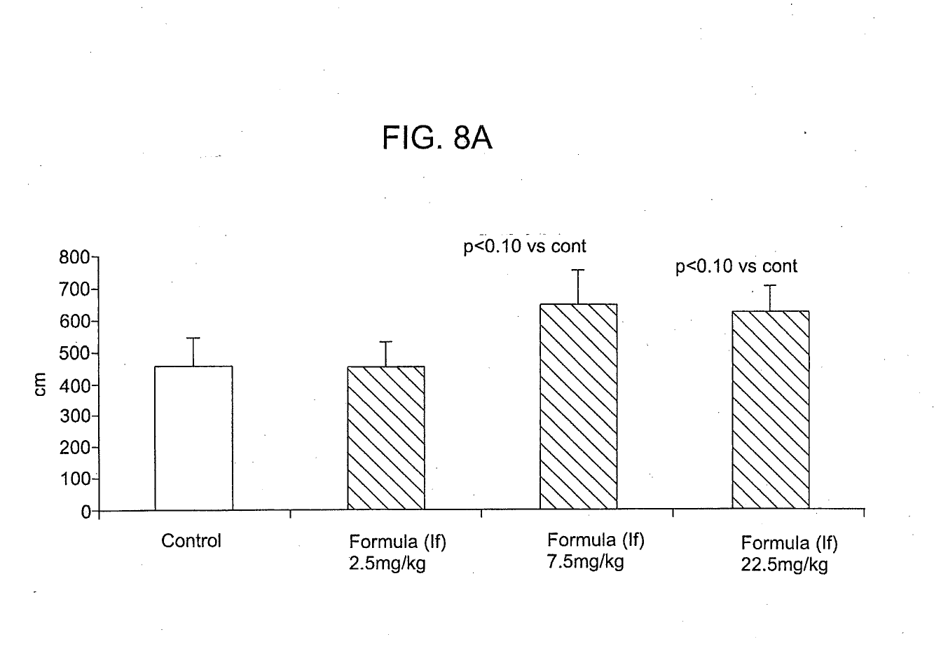

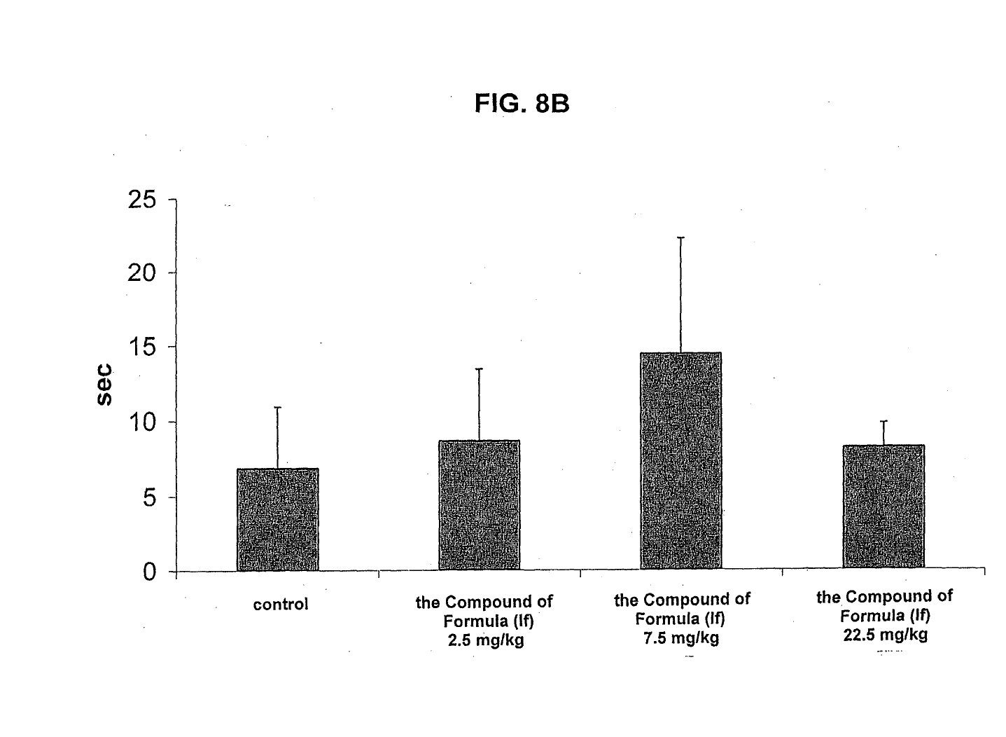

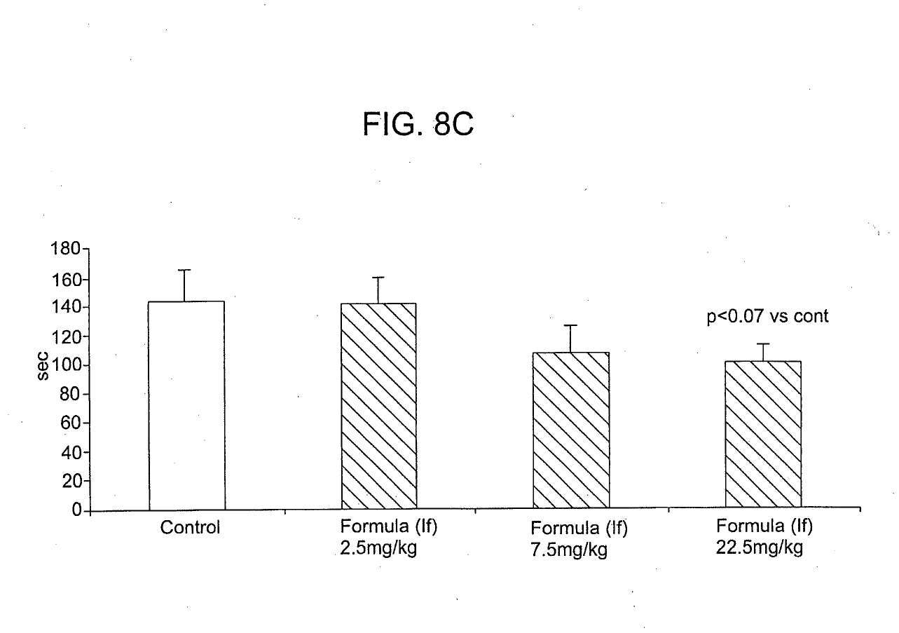

This example demonstrates the effect of an exemplary compound on distance moved, strong mobility, and immobility in the FST in BALB/c mice. FIGS. 8A, 8B, 8C are bars graphs depicting data derived from the administration of the compound of formula (If) to BALB/c mice. The compound of formula (If) was administered ip at 2.5, 7.5 and 22.5 mg/kg and given one hr prior to placing the mice in the water. The results of FIG. 8A show the effect of the compound of formula (If) on distance moved (swimming). The results of FIG. 8B show the effect of the compound of formula (If) on strong mobility, which represents mainly the climbing behavior. The results of FIG. 8C show the effect of the compound of formula (If) on immobility. Each bar represents the mean±SEM of 4 animals.

The data indicate that administration of the compound of formula (If) resulted in increased velocity and distance moved (7.5 and 22.5 mg/kg) and tended to increase strong mobility (7.5 mg/kg). With regard to immobility, a dose dependent decrease in immobility was observed with administration of the compound of formula (If), which was closed to significance (p=0.07) at 22.5 mg/kg, compared to controls. The results suggest that the compound of formula (If) possesses an antidepressant activity.

### Example 13

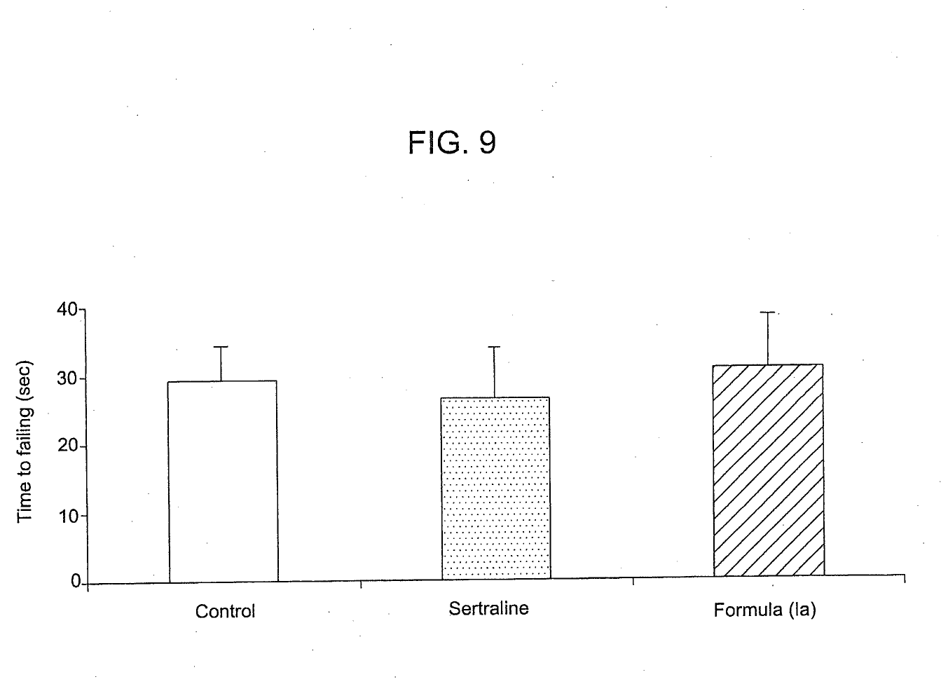

This example demonstrates the effect of an exemplary compound relative to sertraline (control) on motor activity in BALB/c mice. FIG. 9 illustrates motor activity data obtained by the administration of the compound of formula (Ia) and sertraline 10 mg/kg p.o. to BALB/c mice. The test was performed 90 min after oral administration of the compounds. The rotor rod test was designed to test the possible effect of oral sertraline and the compound of formula (Ia) administration on motor activity and muscle strength. The rotor rod test involves placing the mouse on an elevated rotating bar. In this test, the mouse is placed on the bar such that it is facing the direction opposite to the rotation of the rod. The time lag to fall is determined. Each column is the mean±SEM of 12 determinations.

The results indicate that sertraline and the compound of formula (Ia) did not modify muscle strength and basic motor activity in the animals to which the compounds were administered, as compared to the controls.

### Example 14

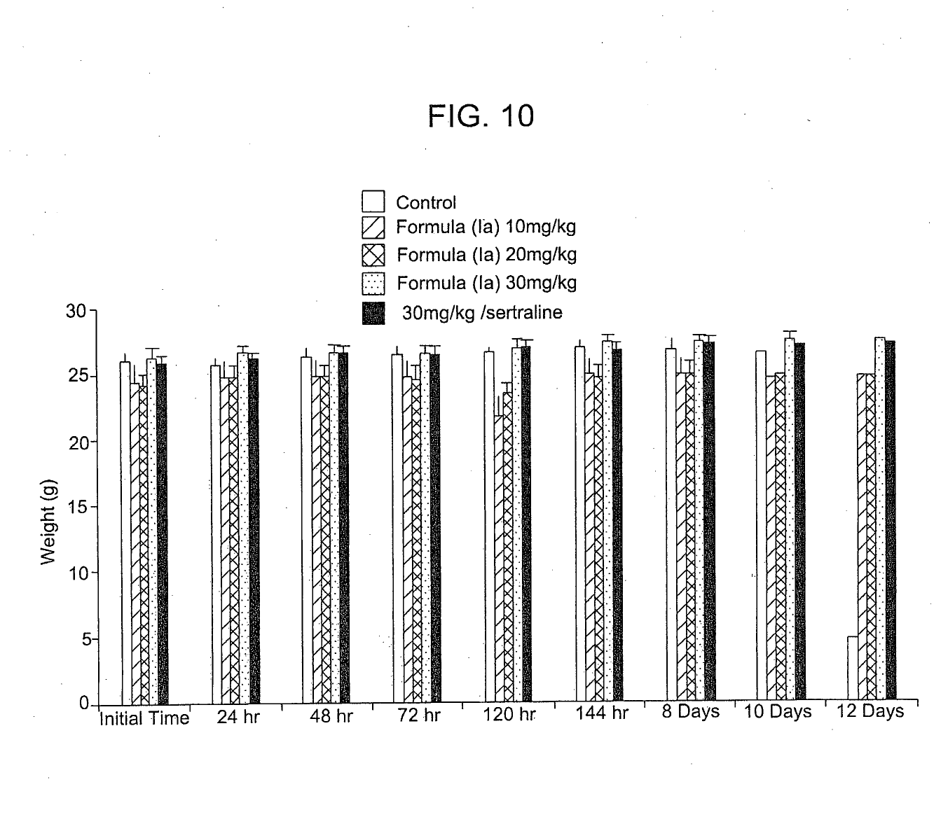

This example demonstrates the effect of an exemplary compound relative to sertraline (control) on body weight in BALB/c mice. FIG. 10 illustrates data demonstrating the effect of the compound of formula (Ia) on the body weight of BALB/c mice, which is a measure of acute toxicity. The compound of formula (Ia) was administered ip to mice at doses of 10, 20 and 30 mg/kg, while sertraline was also administered to one group of mice at 30 mg/kg (5 animals/group). The animals were observed for behavioral changes at 4hr and then after 24 hr. The body weight of the mice was determined up to 12 days after administration of the compounds.

The results show that administration ip of up to 30 mg/kg of the compound of formula (Ia) was well tolerated, since all of the corresponding animals survived. In addition, the animals, both those that received sertraline and those that received the compound of formula (Ia), did not show any changes with regard to behavior, food intake or body weight for up to 12 days.

### Example 15

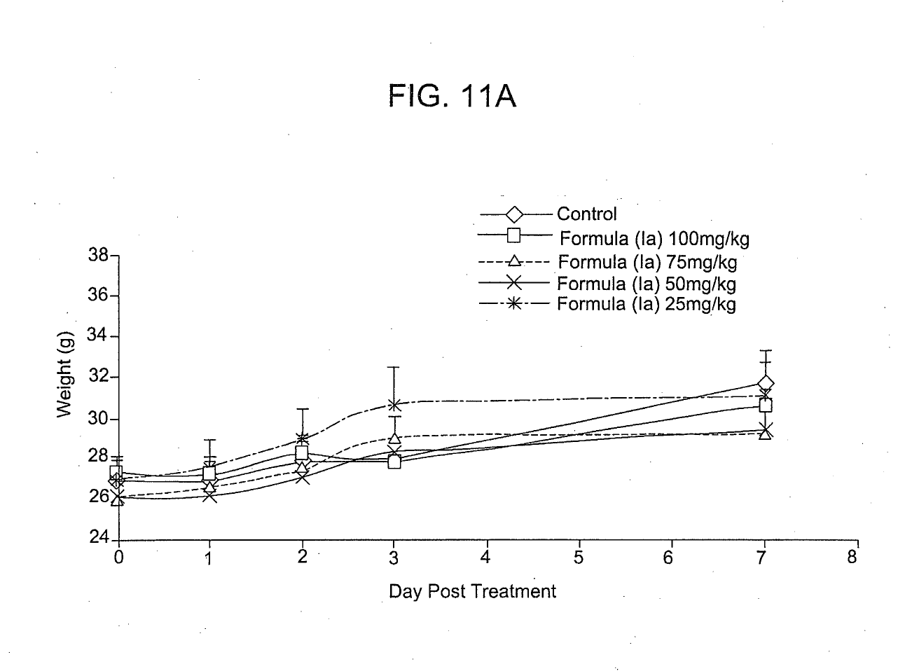

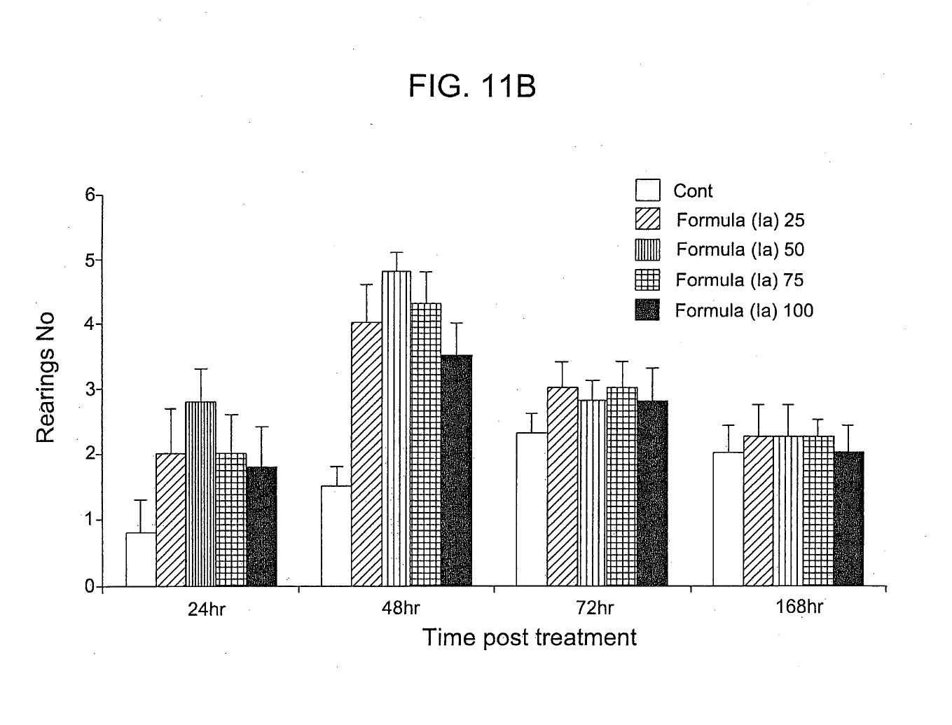

This example demonstrates the effect of an exemplary compound on body weight and stimulation in ICR mice. FIG. 11A illustrates data demonstrating the effect that acute oral administration of the compound of formula (Ia) has on the weight of ICR mice. FIG. 11B illustrates the effect of administering the compound of formula (Ia) on stimulation in ICR mice.

Acute toxicity was assessed in male BALB/c mice that had been orally administered the compound of formula (Ta). The compound of formula (Ia) was administered orally to mice at doses of 20, 25, 75, and 100 mg/kg (4 animals/group). Animals were followed for behavioral changes such as stimulation (number of rearings), and sedation, after 24, 48 and 72 hr. Body weight was determined up to 7 days after drug administration.

The results show that acute oral administration of the compound of formula (Ia) up to 100 mg/kg was well tolerated, all animals survived. All groups showed a similar increase in body weight (A). Animals receiving the compound of formula (Ia) showed a significant increase in stimulation behavior (number of rearings) at 24 and 48hr after drug administration and normalization after 72 hr. (B).

### Example 16

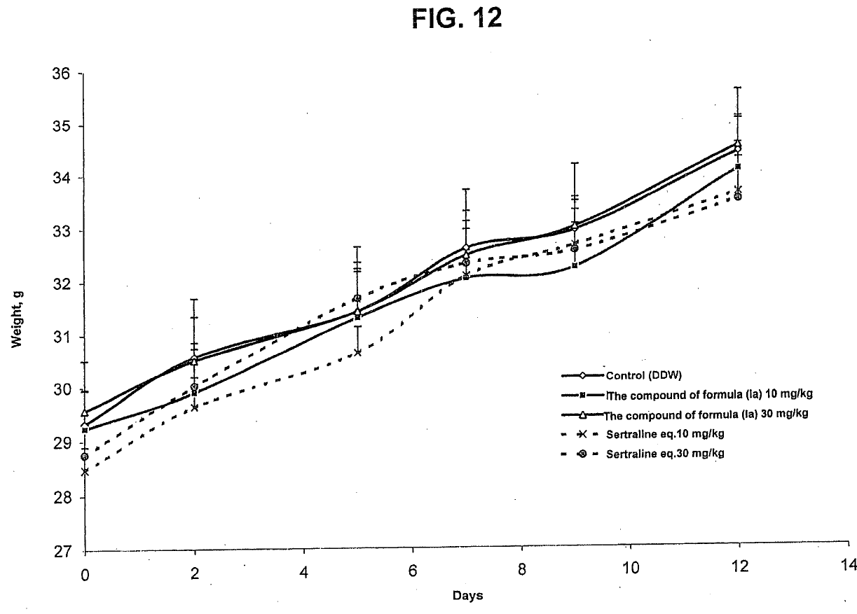

This example demonstrates the effect of an exemplary compound Ia (10 and 30 mg/kg ip ×3/week for 2 weeks) (FIG. 12) and equimolar dose of sertraline on body weight of ICR male mice. The data show that both sertraline and Ia were well tolerated and no difference in body weight gain versus controls was observed. Moreover chemistry blood picture of the drug treated animals did not show a significant change. Histopathology report on organs showed a mild local irritation observed minimal to mild irritation at the capsule of the abdominal organs. Except of these local irritations no other toxicity was noted.

Collectively the results show that the compound of formula (Ia) is well tolerated up to 100 mg/kg when given orally, or up to 30 mg/kg when given ip subchronically. The drug induced a slight stimulation at all doses (highest effect at 25 and 50 mg/kg which lasted up to 48 hr after drug administration.

### Example 17

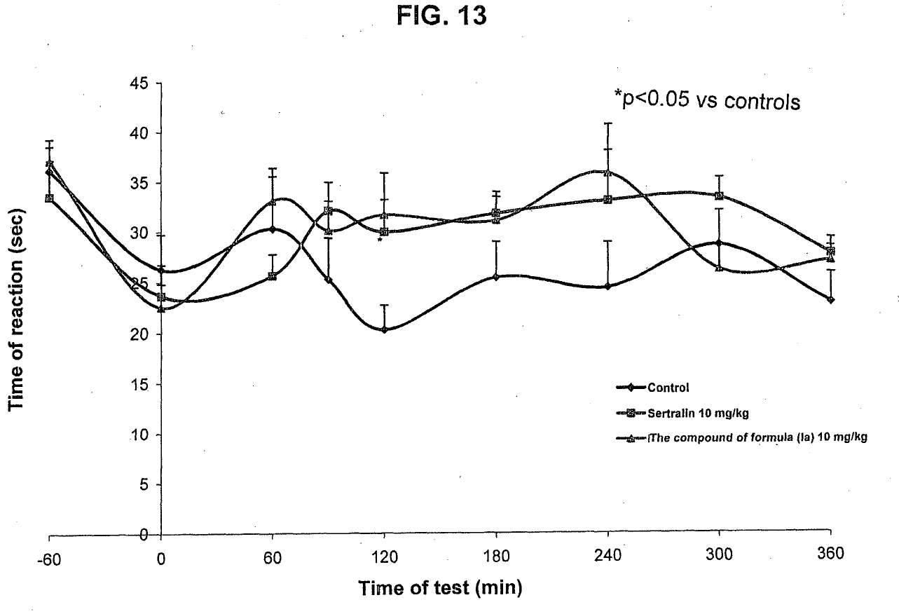

This example demonstrates the effect of an exemplary compound Ia given orally (10 mg/kg) and an equimolar does of sertraline on time to reaction to heating of paw of BALBc male mice on the hot plate (MRC, model-MH-4, 230 V/50 Hz, 750 W maintained at 52-56° C.) (FIG. 13). The animals were followed for 6 hr. The data show that both sertraline and compound Ia significantly increase the latency to heat sensation as compared to control (vehicle treated) animals. Significant delay in reaction to heating was found at 102 min. and 240 min. for compound Ia and at 120 min. following drug administration.

The data show that compound Ia and sertraline significantly inhibited the time of reaction to heating (120 and 240 mins after drug administration for compound Ia and 120 min. for sertaline) suggesting that both compound Ia and sertraline possess a potential analgesic activity and may be used in painful states e.g.: migraine, neuropathich pain, fibromialgia etc.

### Example 18

This example demonstrates the antidepressant activity of an exemplary compound compared to sertraline (control). The compounds associated with the method of the present invention provide antidepressant activity with serotonin reuptake inhibition, dopaminergic uptake inhibition and increased DA activity, as shown below by the data provided in Table 1. Table 1 provides data produced via a receptor binding assay and IC50 and Ki determination. Dopamine transporter, DAT final Ki was 29 nM, Serotonin transporter, SERT final Ki was 1.09 nM. The data provided in Table 1 reveals a potent antidepressant activity with serotonin reuptake inhibition, as shown by the marked inhibition of SERT (Ki of 1.09 nM). In addition, the compound of formula (Ia) has a potent DAT inhibition, which is indicative of dopaminergic uptake inhibition and increased DA activity. In addition to the effect on SERT and DAT, the compound of formula (Ia) inhibited the transporter of Norepinephrine (NET), and several neurotransmitter's receptors like alpha2A and alpha2C receptors, alpha 1A and alphal C, 5HT2A and 5HT2C, signal histamine2 and dopamine D1. This specific profile is in line with the data on antidepressant, anxiolytic and moderate neuro-stimulation activity observed in the animal model studies.

TABLE 1

| | PRIMARY | | | | | | | | | |
|---|---|---|---|---|---|---|---|---|---|---|
| | RADIOLIGAND | | | | | | | | | |
| CAT.# | ASSAY | SPECIES | CONC. | | % INH. | IC₅₀* | | K₁ | | n_{H} |
| 203620 | Adrenergic α_{2A} | hum | 1 | μM | 82 | 0.273 | μM | 0.102 | μM | 0.930 |
| 203800 | Adrenergic α_{2c} | hum | 3 | μM | 62 | 1.9 | μM | 0.276 | μM | 1.03 |
| 203200 | Adrenergic α_{1B} | rat | 1 | μM | 63 | 0.562 | μM | 0.311 | μM | 1.08 |
| 278110 | Sigma σ₁ | hum | 3 | μM | 69 | 0.837 | μM | 0.352 | μM | 0.627 |
| 204410 | Transporter, | hum | 1 | μM | 62 | 0.623 | μM | 0.618 | μM | 0.977 |
| | Norepinephrine (NET) | | | | | | | | | |
| 271650 | Serotonin (5- | hum | 10 | μM | 81 | 3.37 | μM | 0.965 | μM | 1.06 |
| | Hydroxytryptamine) 5- | | | | | | | | | |
| | HT_{2A} | | | | | | | | | |
| 272200 | Serotonin (5- | hum | 3 | μM | 51 | 2.6 | μM | 1.21 | μM | 1.16 |
| | Hydroxytryptamine) 5- | | | | | | | | | |
| | HT₆ | | | | | | | | | |
| 203400 | Adrenergic α_{1D} | hum | 3 | μM | 54 | 2.5 | μM | 1.23 | μM | 1.04 |
| 239710 | Histamine H₂ | hum | 3 | μM | 70 | 1.56 | μM | 1.28 | μM | 1.37 |
| 219500 | Dopamine D₁ | hum | 10 | μM | 84 | 2.7 | μM | 1.35 | μM | 1.25 |
| 203100 | Adrenergic α_{1A} | rat | 10 | μM | 73 | 4.2 | μM | 1.7 | μM | 1.34 |
| 271800 | Serotonin (5- | hum | 10 | μM | 54 | 9.16 | μM | 4.8 | μM | 2.15 |
| | Hydroxytryptamine) 5- | | | | | | | | | |
| | HT_{2C} | | | | | | | | | |
| 220320 | Trasnporter, Dopamine | hum | 0.03 | μM | 51 | <0.3 | μM | | | |
| | (DAT) | | | | | | | | | |
| 274030 | Transporter, Serotonin | hum | 0.03 | μM | 86 | <0.3 | μM | | | |
| | (5-Hydroxytryptamine) | | | | | | | | | |
| | (SERT) | | | | | | | | | |

With regard to the data provided in Table 1, the biochemical assay results are presented as the percent inhibition of specific binding or activity throughout the report. For primary assays, only the lowest concentration with a significant response judged by the assays’ criteria, is shown in this summary. Where applicable, either the secondary assay results with the lowest dose/concentration meeting the significance criteria or, if inactive, the highest dose/concentration that did not meet the significance criteria is shown. Unless otherwise requested, primary screening in duplicate with quantitative data (e.g., IC50±SEM, Ki±SEM and nH) are shown.

The receptor binding profile resembles that of sertraline with small differences e.g., Ki for SERT of sertraline is 0.47 nM and DAT Ki is 0.026 μM. These findings suggest that compound Ia preserves the potent SERT and DAT activity of sertraline.

### Example 19

This example demonstrates the effect of an exemplary compound (Ia) as compared to other SSRIs:sertraline (sert), desmethyl sertraline (Des-ser) and paroxetine (Par) on cell viability 24 hours and 48 hours after incubation with human neurons from a neuroblastoma —SHSY5Y cell-line at a concentration range of 1-50 μM. A biphasic effect of all agents was demonstrated with low concentrations inducing a neurotrophic effect and at high concentrations inducing apoptosis.

Table 2 demonstrates the percentage of viable cells (as percent of controls) at different concentrations. The grey bars represent the neuroprotective/neurotrophic activity, and the white bars represent the apoptotic effects. Each point is the mean of a triplicate using the neutral red method for cell viability.

TABLE 2

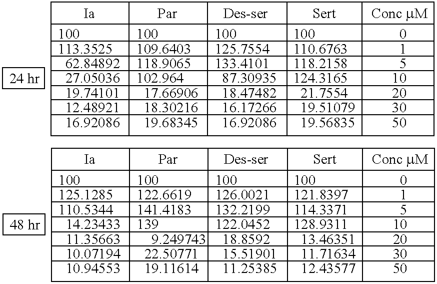

The results show that compound Ia at concentrations of 1-5 μM increases cell viability by 13-25% compared to control vehicle treated cells. The low concentrations actually correspond to the blood concentrations after antidepressant activity. Compound 1a showed biphasic effects like all other SSRIs, yet, its neurotropic activity was maximal at 1 μM while other compounds reached a peak at 5 and 10 μM.
Conclusion: The results suggest a potential neuroprotective effect of compound 1a at concentrations relevant to the psychiatric efficacy.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for treating or preventing a disease or disorder treatable by the inhibition of serotonin reuptake in a patient, and/or norepinephrine reuptake and/or dopamine reuptake in a patient, the method comprising administering to the patient a neurotransmitter reuptake inhibiting-effective amount of at least one compound of the formula A-L-B (I), wherein:
A is selected from the group consisting of: 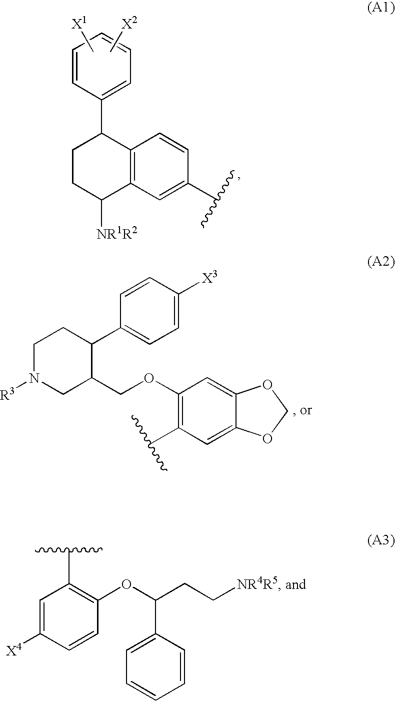
wherein:
R¹, R², R³, R⁴ and R⁵ are the same or different and each is independently a hydrogen or a C₁₋₆ alkyl,
X¹ and X² are the same or different and each is independently a hydrogen, a halogen, a C₁₋₆ haloalkyl, a C₁₋₆ alkoxy, or a cyano,
X³ is a hydrogen, a C₁₋₆ alkyl, a C₁₋₆ alkoxy, a C₁₋₆ haloalkyl, a hydroxyl, a halogen, a C₁₋₆ alkylthio, or an aryl(C₁₋₆)alkoxy, and
X⁴ is a halogen, a C₁₋₆ haloalkyl, a C₁₋₆ alkyl, a C₁₋₆ alkoxy, or a C₂₋₆ alkenyl;
L is a linking group comprising two carbon atoms; and
B is an alkyl, alkenyl, alkynyl or aralkyl comprising at least one substituent of the formula Q, wherein:
the alkyl, alkenyl, alkynyl or aralkyl is optionally substituted with one or more halogens, hydroxyl, cyano, nitro, amino, or thiol; and
Q is OR⁶, OC(O)R⁶, C(O)R⁶, C(S)R⁶, CO₂R⁶, C(O)SR⁶, C(O)NR⁶R⁷, C(S)NR⁶R⁷, NR⁶R⁷, NR⁶C(O)R⁷, NR⁶C(S)R⁷, NR⁶C(O)NR⁷R⁸, NR⁶C(S)NR⁷R⁸, NR⁶SO₂R⁷, NR⁶SO₂NR⁷R⁸, SR⁶, SC(O)R⁶, SC(O)NR⁶R⁷, S(O)R⁶, SO₂R⁶, SO₂NR⁶R⁷, or NR⁶SO₂NR⁷R⁸, wherein R⁶, R⁷, and R⁸ are the same or different and each is independently a hydrogen, a C₁₋₆ alkyl, an aryl, an aralkyl, or a pharmaceutically acceptable solubility modifying group;
or a salt, ester, or prodrug thereof.

2. The method of claim 1, wherein B is —(CH₂)ₙ(CHR⁹)ₘQ,
—Ar(CH₂)ₙ(CHR⁹)ₘQ, —(CH₂)ₙAr(CHR⁹)ₘQ or —(CH₂)ₙ(CHR⁹)ₘArQ, wherein m and n are the same or different and each is independently from 0 to about 6 provided that m and n are not both zero when B is —(CH₂)ₙ(CHR⁹)ₘQ; Ar is a bivalent aryl; R⁹ is a hydrogen, a C₁₋₆ alkyl, or an aryl; and the Ar, (CH₂)ₙ and (CHR⁹)ₘ are optionally substituted with one or more halogens, hydroxyl, cyano, nitro, amino, or thiol.

3. The method of claim 1, wherein B is —(CH₂)ₙQ, —(CH₂)ₙArQ or
—Ar(CH₂)ₙQ, wherein n is from 0 to about 6 provided that n is not zero when B is —(CH₂)ₙQ.

4. The method of claim 3, wherein B is —(CH₂)ₙQ.

5. The method of claim 1, wherein n is about 3.

6. The method of claim 1, wherein Q is OR⁶, OC(O)R⁶, NR⁶R⁷, SR⁶ or SC(O)R⁶.

7. The method of claim 1, wherein A is represented by formula (A1).

8. The method of claim 7, wherein X¹ and X² are the same or different and each is a halogen.

9. The method of claim 7, wherein R¹ and R² are the same or different and each is independently a hydrogen or a methyl.

10. The method of claim 7, wherein (A1) is represented by the formula: 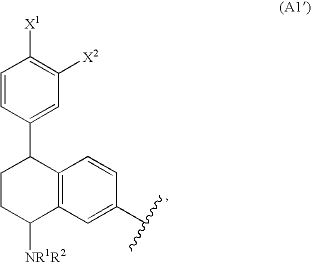
wherein X¹ and X² are the same or different and each is a halogen, and R¹ and R² are the same or different and each is independently a hydrogen or a methyl.

11. The method of claim 10, wherein X¹ and X² are chlorine, and one of R¹ and R² is a hydrogen and the other is methyl.

12. The method of claim 1, wherein A is represented by formula (A2).

13. The method of claim 12, wherein R³ is a hydrogen.

14. The method of claim 12, wherein X³ is a halogen.

15-16. (canceled)

17. The method of claim 1, wherein L comprises a carbon-carbon single bond, a carbon-carbon double bond, or a carbon-carbon triple bond.

18. (canceled)

19. The method of claim 1, wherein R⁶, R⁷, and R⁸ are hydrogen.

20. The method of claim 2, wherein n is from 2 to 4.

21. The method of claim 1, wherein the compound is of the formula: 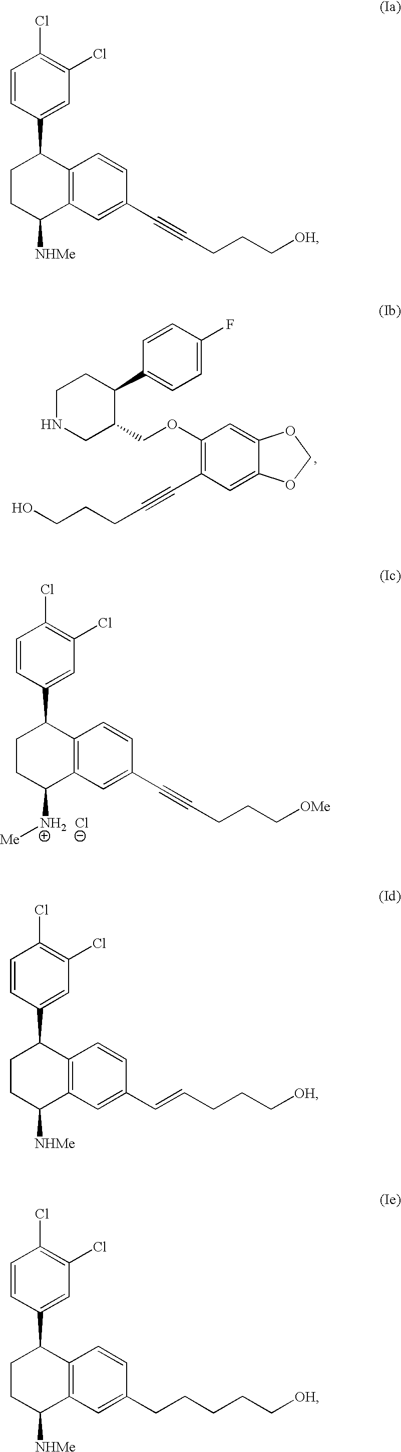 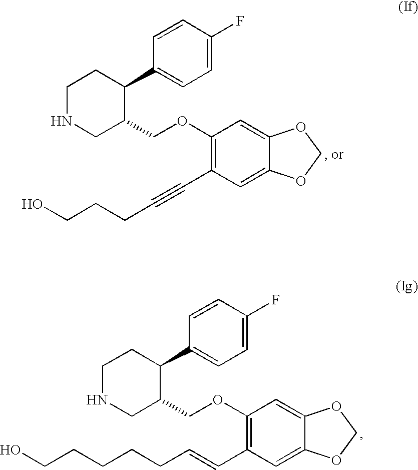
or a pharmaceutically acceptable salt, ester or prodrug thereof.

22-24. (canceled)

25. The method of claim 1, wherein the disease or disorder is selected from the group consisting of a psychiatric disease or disorder, a disease or disorder associated with cancer in the patient and a disease or disorder associated with abnormal serotonin uptake.

26. (canceled)

27. The method of claim 25, wherein the disease or disorder is selected from the group consisting of major depressive disorder, social anxiety disorder, obsessive compulsive disorder (OCD), panic disorder (PD), generalized anxiety disorder (GAD), posttraumatic stress disorder (PTSD), bulimia nervosa, premenstrual dysphoric disorder (PMDD), premature ejaculation, arthritis, chronic fatigue, multiple sclerosis, lupus, irritable bowel syndrome (IBS), migraine headache, diabetic neuropathy, fibromyalgia, attention-deficit/hyperactivity disorder (ADHD), autistic spectrum disorders, bipolar depression, attention deficit disorder, chronic pain, neurocardiogenic syncope, post traumatic stress disorders, obsessive compulsive disorders, anxiety, panic attacks, pain, neuralgic pain, postherpetic neuralgia, phobias of various types, and eating disorders.

28-33. (canceled)
